(19) United States
(12) Patent Application Publication
(10) Pub. No.: US 2012/0114588 A1
(43) Pub. Date: May 10, 2012

(54) IBAT INHIBITORS FOR TREATMENT OF METABOLIC DISORDERS AND RELATED CONDITIONS

(75) Inventors: Ingemar Starke, Goteborg (SE); Hans Graffner, Helsingborg (SE); Per-Göran Gillberg, Molndal (SE); Ann-Margret Lindqvist, Askim (SE); Bo Angelin, Stockholm (SE)

(73) Assignee: ALBIREO AB, Goteborg (SE)

(21) Appl. No.: 13/116,422

(22) Filed: May 26, 2011

Related U.S. Application Data
(60) Provisional application No. 61/410,963, filed on Nov. 8, 2010.

(51) Int. Cl. A61K 31/554; A61K 31/785; A61K 38/26; C07D 285/36; A61P 1/04; A61P 3/10; A61P 9/00; A61P 29/00; A61P 1/00; C07D 281/10; A61P 3/04

(52) U.S. Cl. 424/78.01; 540/552; 514/211.09; 514/7.2; 514/5.3; 540/545; 514/211.08

(57) ABSTRACT

The present invention regards specific IBAT inhibitors with improved effect in prophylaxis and treatment of metabolic syndrome, obesity, disorders of fatty acid metabolism, glucose utilization disorders, disorders in which insulin resistance is involved, diabetes mellitus, type 1 and type 2 diabetes.

It also relates to compositions comprising these IBAT inhibitors, a method for treatment of the disorders and a kit comprising the substances or the compositions.

### CROSS-REFERENCE TO RELATED APPLICATIONS

This application claims priority under 35 U.S.C. §119(e) to U.S. Provisional Application No. 61/410,963, filed Nov. 8, 2010, which is incorporated by reference herein in its entirety.

### BACKGROUND OF THE INVENTION

Obesity is a medical condition affecting numerous humans in a number of countries throughout the world, and is associated with or induces other diseases or conditions. In particular, obesity is a serious risk factor for diseases and conditions such as diabetes, hypertension, gallbladder disease, cancer, polycystic ovary disease and arteriosclerosis and can contribute to elevated levels of cholesterol in the blood. In addition, increased body weight due to obesity places a burden on joints causing arthritis, pain and stiffness. Overeating and obesity have become a problem in the general population. Consequently there is interest in reducing food intake, losing weight, reducing weight, and/or maintaining a healthy body weight and lifestyle.

WO10062861 and WO 2008/058630 describe the effect of ileal bile acid transport (IBAT) in the treatment of obesity and diabetes.

### SUMMARY OF THE INVENTION OF THE INVENTION

The present invention regards specific ileal bile acid transport (IBAT) inhibitors with improved effect in prophylaxis and treatment of metabolic syndrome, obesity, disorders of fatty acid metabolism, glucose utilization disorders, disorders in which insulin resistance is involved, diabetes mellitus, type 1 and type 2 diabetes. It also relates to compositions comprising these IBAT inhibitors, a method for treatment of the disorders and a kit comprising the substances or the compositions. It has turned out that Interruption of bile acid circulation in mice improves triglyceride metabolism and normalizes elevated plasma glucose levels.

### FIGURE LEGENDS

FIG. 1

(A) Schematic overview of vector and strategy used to target the Slc10a2 wt allele in order to obtain a Slc10a2 null mouse.

(B) A representative immunoblot employing a specific antibody directed against the Slc10a2 protein demonstrates absence of Slc10a2 protein expression in ileum of Slc10a2−/− mice.

FIG. 2

Disruption of the ileal BA transporter gene Slc10a2 in mice activates enzymes involved in BA synthesis and suppresses mRNA levels of the hepatic orphan nuclear receptor SHP. (A) mRNA levels 11 of CYP7A1 in livers of wt, Slc10a2+/− and Slc10a2−/− animals. (B) CYP7A1 enzymatic activity in pooled microsomal samples from livers of wt, Slc10a2+/− and Slc10a2−/− animals. Results shown are mean of two analyses. (C) Protein levels of CYP7A1 in livers of wt, Slc10a2+/− and Slc10a2−/− mice determined in pooled microsomal samples by immunoblot using a CYP7A1 specific antibody. 20 pg and 40 pg of protein were used per sample, respectively. The results are representative of three separate immunoblots. Note the loading order of the samples. (D) Serum levels of the CYP7A1 activity marker product C4 were assayed as an indirect measure of CYP7A1 enzymatic activity in pooled serum samples of wt, Slc10a2+/− and Slc10a2−/− animals. Data show mean of two separate measurements. (E) hepatic mRNA levels of CYP8B1, and (F) hepatic SHP mRNA levels of wt, Slc10a2+/− and Slc10a2−/− mice. mRNA levels for the wt mice were normalized to 1.

Data are expressed as mean ±standard error (SEM) for the mRNA analysis. wt littermates (n=10), Slc10a2+/− (n=9) and Slc10a2−/− (n=10) for A-F. A p-value <0.05 is denoted *, p<0.01 is denoted **.

FIG. 3

Ablation of the Slc10a2 gene leads to lowered levels of plasma TGs but not plasma cholesterol with concurrent alterations of expression levels of genes involved in hepatic sterol and TG homeostasis. (A) Total plasma cholesterol and TGs from wt, Slc10a2+/− and Slc10a2−/− mice. (B) Plasma profiles of cholesterol and TGs (C) were analysed from wt, Slc10a2+/− and Slc10a2−/− animals by FPLC. Lines represent means of all individuals from each group, respectively. Lipoprotein fractions are indicated, n=9-10. (D) Hepatic HMGCoA reductase mRNA levels and enzymatic activity of wt, Slc10a2+/− and Slc10a2−/− mice. HMGCoA reductase enzymatic activity was analysed from pooled hepatic microsomal samples and represents mean of two measurements. (n=9-10). (E) Hepatic mRNA levels of the sterol transporters ABCG5 and ABCG8 in wt, Slc10a2+/− and Slc10a2−/− mice. (F) Hepatic mRNA levels of SREBP1 c and SREBP2, in wt, Slc10a2+/− and Slc10a2−/− mice. mRNA levels in wt mice were normalized to 1. Data are expressed as mean±standard error (SEM) for the mRNA and total plasma cholesterol and TGs analysis. wt littermates (n=10), Slc10a2+/− (n=9) and Slc10a2−/− (n=10) for A-F. A p-value<0.05 is denoted *.

FIG. 4

Slc10a2−/− mice display lower hepatic TG and cholesterol accumulation than wild type mice concomitant with reduced expression of fatty acid synthesis genes following a sucrose-rich diet. (A) Liver TG and cholesterol content were analysed from a total of four groups; wt and Slc10a2−/− mice fed either regular chow or a sucrose-rich diet (SR) for a period of two weeks. (B) Hepatic mRNA levels of enzymes involved in fatty acid synthesis from wt or Slc10a2−/− animals, as in (A). (C) SREBP1 immunoblots were performed on pooled liver cytoplasmic and nuclear protein preparations, respectively, from wt or Slc10a2−/− mice fed a regular chow or a sucrose-rich (SR) diet using an antibody specific for the N′-terminus of SREBP1. An antibody against Lamin was used as nuclear loading control. The displayed blot is representative of three separate immunoblots. Hepatic mRNA expression levels of (D) Glucokinase, (GK); pyruvate kinase, (LPK); and (E) Glucose-6 phosphodehydrogenase, (G6PDH) and Malic enzyme, (ME), mRNA values in the wt group fed regular chow was normalized to 1. Data are expressed as mean±standard error (SEM) for the mRNA, hepatic cholesterol and TG analysis. wt (n=6), Slc10a2−/− (n=5), wt+SR (n=6) and Slc10a2+/− SR (n=6) for A-D. A p-value<0.05 is denoted *.

FIG. 5

Interruption of BA circulation by inhibition of the Slc10a2 protein improves plasma glucose and TGs in ob/ob mice. Ob/ob mice were treated with a specific Slc10a2 protein inhibitor (Example 14) , or control vehicle, (vehicle) (see experimental procedures). (A) fasting levels of blood glucose and plasma insulin, (B) plasma total TGs and cholesterol. (C) Hepatic mRNA levels of FGF21 and CYP7A1. (D) Liver cholesterol and TGs (E) Distal ileum mRNA levels of FGF15, SHP, IBABP and FXR from ob/ob animals treated with a Slc10a2 protein inhibitor. mRNA levels of the control vehicle treated animals are normalized to 1. Data are represented as mean±standard error (SEM). A p-value<0.05 is denoted *. P<0.01 is denoted **.

FIG. 6

Pharmacological inhibition of Slc10a2 leads to reduced hepatic SREBP1c mRNA and altered levels of glucose metabolic enzymes levels in ob/ob mice. Ob/ob mice were treated with the specific Slc10a2 protein inhibitor, Example 14, (IBAT inhibitor) or control vehicle, (Controls) (see experimental procedures). (FIG. 6A) Hepatic mRNA levels of SREBP1c, and its target genes ACC, FAS, and SCD1. (B) Hepatic mRNA levels of GK, LPK, G6Pase and PEPCK. mRNA levels of the control vehicle treated animals are normalized to 1. Data are represented as mean±standard error (SEM). A p-value<0.05 is denoted *.

FIG. 7

Pharmacological inhibition of Slc10a2 induces altered activity of important signal transduction pathways in ob/ob liver. The activation state of selected kinases important in glucose and lipid metabolism were investigated in individual liver protein extracts by phosphorylation site-specific antibodies in Slc10a2 inhibitor treated and control ob/ob mice. (A) western blot against liver pAkt (ser 473), the same membrane was stripped and reprobed with an antibody against total amount of Akt, (B) western blot against liver pMek1/2 (ser 217/221) and pErk1/2 Thr 202/ Tyr 204), the same membranes were consecutively stripped and reprobed with an antibodies against total Mek1/2 and Erk1/2, (C) western blot against liver pAmpk (Thr 172), the same membrane was stripped and reprobed with an antibody against total amount of Ampk. All membranes were finally reprobed with an antibody against beta-actin. Blots are representative of four individual runs.

FIG. 8

Percentage change of plasma glucose in patients with glucoseUpper Limit normal (ULN) (Change from Baseline—End of Treatment, completer population), 10 mg of Example 14 versus placebo p=0.013.

### DETAILED DESCRIPTION OF THE INVENTION

The invention relates to compounds having IBAT inhibitory effect chosen of formula (I):

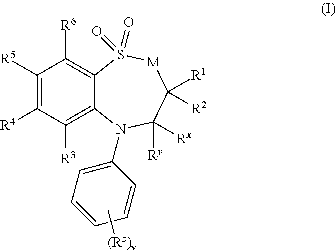

wherein:

M is CH₂, NH

One of R¹ and R² are selected from hydrogen or C₁₋₆alkyl and the other is selected from C₁₋₆alkyl;

R^{x} and R^{y} are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2

R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O), wherein a is 0 to 2, C₁₋₆alkoxycarbonyl, N-(C₁₋₆alkyl)sulphamoyl and N,N-(C₁₋₆alkyl)₂sulphamoyl;

v is 0-5;

one of R⁴ and R⁵ is a group of formula (IA):

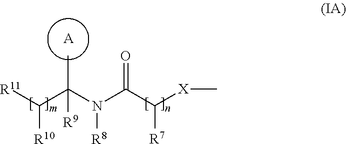

R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;

X is —O—, —N(R^{a})—, —S(O)_{b}— or —CH(R^{a})—; wherein R² is hydrogen or C₁₋₆alkyl and b is 0-2;

Ring A is aryl or heteroaryl; wherein Ring A is optionally substituted by one or more substituents selected from R¹⁷;

R⁷ is hydrogen, C₁₋₄alkyl, carbocyclyl or heterocyclyl; wherein R⁷ is optionally substituted by one or more substituents selected from R¹⁸;

R⁸ is hydrogen or C₁₋₄alkyl;

R⁹ is hydrogen or C₁₋₄alkyl;

R¹⁰ is hydrogen, C₁₋₄alkyl, carbocyclyl or heterocyclyl; wherein R¹⁰ is optionally substituted by one or more substituents selected from R¹⁹;

R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (IB) or (IC):

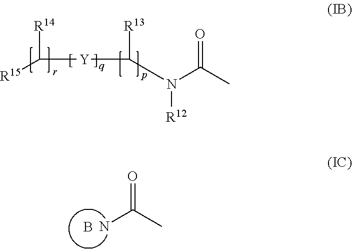

wherein:

Y is —N(Rn)—, —N(Rⁿ)C(O)—, —N(Rⁿ)C(O)(CR^{s}R^{t})ᵥN(Rⁿ)C(O)—, —O—, and —S(O)a—; wherein a is 0-2, v is 1-2, R^{s} and R^{t} are independently selected from hydrogen or C₁₋₄alkyl optionally substituted by R²⁶ and Rⁿ is hydrogen or C₁₋₄alkyl;

R¹² is hydrogen or C₁₋ₐalkyl; R¹³ and R¹⁴ are independently selected from hydrogen, C₁₋₄alkyl, carbocyclyl or heterocyclyl; and when q is 0, R¹⁴ may additionally be selected from hydroxy wherein R¹³ and R¹⁴ K may be independently optionally substituted by one or more substituents selected from R²⁰;

R¹⁵ is carboxy, sulpho, sulphino, phosphono, —P(O)(OR^{e})(OR^{f}), —P(O)(OH)(OR^{e}), —P(O)(OH)(R^{e}) or —P(O)(OR^{e})(R^{f}) wherein R^{e} and R^{t} are independently selected from C₁₋₆alkyl;

p is 1-3; wherein the values of R¹³ may be the same or different; q is 0-1;

r is 0-3; wherein the values of R¹⁴ may be the same or different; m is 0-2; wherein the values of R¹⁰ may be the same or different; n is 1-3; wherein the values of R⁷ may be the same or different; Ring B is a nitrogen linked heterocyclyl substituted on carbon by one group selected from R²³, and optionally additionally substituted on carbon by one or more R²⁴; and wherein if said nitrogen linked heterocyclyl contains an —NH—moiety, that nitrogen may be optionally substituted by a group selected from R25;

R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino,

N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl and N,N—(C₁₋₄alkyl)₂sulphamoyl; wherein R¹⁶, R¹⁷ and R¹⁸ may be independently optionally substituted on carbon by one or more R²¹;

R¹⁹, R²⁰, R²⁴ and R²⁶ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, carbocyclyl, heterocyclyl, benzyloxycarbonylamino, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹, R²⁰, R²⁴ and R²⁶ may be independently optionally substituted on carbon by one or more R²²;

R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N—methylcarbamoyl, N,N—dimethylcarbamoyl, methylthio, methylsulphinyl, mesyl, N—methylsulphamoyl and N,N—dimethylsulphamoyl;

R²³ is carboxy, sulpho, sulphino, phosphono, —P(O)(OR^{g})(OR^{h}), —P(O)(OH)(OR^{g}), —P(O)(OH)(R^{g}) or —P(O)(OR^{g})(R^{b}) wherein R^{g} and R^{b} are independently selected from C₁₋₆alkyl;

R²⁵ is selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

or a pharmaceutically acceptable salt, solvate, solvate of such a salt or a prodrug thereof for use in prophylaxis and treatment of metabolic syndrome, obesity, disorders of fatty acid metabolism, glucose utilization disorders, disorders in which insulin resistance is involved, diabetes mellitus, type 1 and type 2 diabetes.

It has turned out that the IBAT inhibitors of the present invention reduce the activity of two major kinases namely Akt and Mek1/2. It is known that the Akt and Mek1/2-Erk1/2 pathways are important in hepatic regulation of both glucose metabolism and lipogenesis was examined.

The kinase Akt has been demonstrated to be a crucial component in regulating the hepatic response to insulin and other circulating factors with capacity to favour glycolysis and lipogenesis, and inhibit gluconeogenesis upon food intake.

The Mek1/2-Erk1/2 pathway is activated by the insulin receptor and the FGF receptor 4/beta-Klotho complex, known as the FGF15 receptors.

Thus, the IBAT inhibitors of the present invention has improved efficacy on metabolic syndrome, obesity, disorders of fatty acid metabolism, glucose utilization disorders, disorders in which insulin resistance is involved, diabetes mellitus, type 1 and type 2 diabetes.

Further, the substances turned out to be very specifically effective against high glucose values in serum, whereas normal values were almost not affected. Consequently, the risk of hypoglycemi is minimal.

In the literature IBAT inhibitors are often referred to by different names. It is to be understood that where IBAT inhibitors are referred to herein, this term also encompasses compounds known in the literature as: i) ileal apical sodium co-dependent bile acid transporter (ASBT) inhibitors; ii) bile acid transporter (BAT) inhibitors; iii) ileal sodium/bile acid cotransporter system inhibitors; iv) apical sodium-bile acid cotransporter inhibitors; v) ileal sodium-dependent bile acid transport inhibitors; vi) bile acid reabsorption (BARI's) inhibitors; and vii) sodium bile acid transporter (SBAT) inhibitors; where they act by inhibition of IBAT.

In this specification the term “alkyl” includes both straight and branched chain alkyl groups but references to individual alkyl groups such as “propyl” are specific for the straight chain version only. For example, “C₁₋₆alkyl” includes C₁₋₄alkyl, C₁₋₃alkyl, propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals, for example “phenylC₁₋₆alkyl” would include phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

“Heteroaryl” is a totally unsaturated, mono or bicyclic ring containing 3-12 atoms of which at least one atom is chosen from nitrogen, sulphur or oxygen, which may, unless otherwise specified, be carbon or nitrogen linked. Preferably “heteroaryl” refers to a totally unsaturated, monocyclic ring containing 5 or 6 atoms or a bicyclic ring containing 9 or 10 atoms of which at least one atom is chosen from nitrogen, sulphur or oxygen, which may, unless otherwise specified, be carbon or nitrogen linked. In another aspect of the invention, “heteroaryl” refers to a totally unsaturated, monocyclic ring containing 5 or 6 atoms or a bicyclic ring containing 8, 9 or 10 atoms of which at least one atom is chosen from nitrogen, sulphur or oxygen, which may, unless otherwise specified, be carbon or nitrogen linked. Examples and suitable values of the term “heteroaryl” are thienyl, isoxazolyl, imidazolyl, pyrrolyl, thiadiazolyl, isothiazolyl, triazolyl, pyranyl, indolyl, pyrimidyl, pyrazinyl, pyridazinyl, pyridyl and quinolyl. Preferably the term “heteroaryl” refers to thienyl or indolyl.

“Aryl” is a totally unsaturated, mono or bicyclic carbon ring that contains 3 -12 atoms.

Preferably “aryl” is a monocyclic ring containing 5 or 6 atoms or a bicyclic ring containing 9 or 10 atoms. Suitable values for “aryl” include phenyl or naphthyl. Particularly “aryl” is phenyl.

A “heterocyclyl” is a saturated, partially saturated or unsaturated, mono or bicyclic ring containing 3 -12 atoms of which at least one atom is chosen from nitrogen, sulphur or oxygen, which may, unless otherwise specified, be carbon or nitrogen linked, wherein a —CH₂-group can optionally be replaced by a —C(O)— or a ring sulphur atom may be optionally oxidised to form the S-oxides. Preferably a “heterocyclyl” is a saturated, partially saturated or unsaturated, mono or bicyclic ring containing 5 or 6 atoms of which at least one atom is chosen from nitrogen, sulphur or oxygen, which may, unless otherwise specified, be carbon or nitrogen linked, wherein a —CH₂-group can optionally be replaced by a —C(O)— or a ring sulphur atom may be optionally oxidised to form S-oxide(s). Examples and suitable values of the term “heterocyclyl” are thiazolidinyl, pyrrolidinyl, pyrrolinyl, 2-pyrrolidonyl, 2,5-dioxopyrrolidinyl, 2-benzoxazolinonyl, 1,1-dioxotetrahydrothienyl, 2,4-dioxoimidazolidinyl, 2-oxo-1,3,4-(4-triazolinyl), 2-oxazolidinonyl, 5,6-dihydrouracilyl, 1,3-benzodioxolyl, 1,2,4-oxadiazolyl, 2-azabicyclo [2.2.1] heptyl, 4-thiazolidonyl, morpholino, 2-oxotetrahydrofuranyl, tetrahydrofuranyl, 2,3-dihydrobenzofuranyl, benzothienyl, tetrahydropyranyl, piperidyl, 1-oxo-1,3-dihydroisoindolyl, piperazinyl, thiomorpholino, 1,1-dioxothiomorpholino, tetrahydropyranyl, 1,3-dioxolanyl, homopiperazinyl, thienyl, isoxazolyl, imidazolyl, pyrrolyl, thiadiazolyl, isothiazolyl, 1,2,4-triazolyl, 1,3,4-triazolyl, pyranyl, indolyl, pyrimidyl, thiazolyl, pyrazinyl, pyridazinyl, pyridyl, 4-pyridonyl, quinolyl and 1-isoquinolonyl.

A “carbocyclyl” is a saturated, partially saturated or unsaturated, mono or bicyclic carbon ring that contains 3 -12 atoms; wherein a —CH₂-group can optionally be replaced by a —C(O)—. Preferably “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. Particularly “carbocyclyl” is cyclopropyl, cyclobutyl, 1-oxocyclopentyl, cyclopentyl, cyclopentenyl, cyclohexyl, cyclohexenyl, phenyl or 1-oxoindanyl.

An example of “C₁₋₆alkanoyloxy” and “C₁₋₄alkanoyloxy” is acetoxy. Examples of “C₁₋₆alkoxycarbonyl” and “C₁₋₄alkoxycarbonyl” include methoxycarbonyl, ethoxycarbonyl, n- and t-butoxycarbonyl. Examples of “C₁₋₆alkoxy” and “C₁₋₄alkoxy” include methoxy, ethoxy and propoxy. Examples of “C₁₋₆alkanoylamino” and “C₁₋₄alkanoylamino” include formamido, acetamido and propionylamino. Examples of “C₁₋₆alkylS(O)ₐ wherein a is 0 to 2” and “C₁₋₄alkylS(O)ₐ wherein a is 0 to 2” include methylthio, ethylthio, methylsulphinyl, ethylsulphinyl, mesyl and ethylsulphonyl. Examples of “C₁₋₆alkanoyl” and “C₁₋₄alkanoyl” include C₁₋₃alkanoyl, propionyl and acetyl. Examples of “N-(C₁₋₆alkyl)amino” and “N-(C₁₋₄alkyl)amino” include methylamino and ethylamino. Examples of “N,N-(C₁₋₆alkyl)₂amino” and “N,N-(C₁₋₄alkyl)₂amino” include di—N-methylamino, di-(N-ethyl)amino and N-ethyl—N-methylamino. Examples of “C₂₋₆alkenyl” and “C₂₋₄alkenyl” are vinyl, allyl and 1-propenyl. Examples of “C₂-₆alkynyl” and “C₂₋₄alkynyl” are ethynyl, 1-propynyl and 2-propynyl. Examples of “N-(C₁₋₆alkyl)sulphamoyl” and “N-(C₁₋₄alkyl)sulphamoyl” are N-(C₁₋₃alkyl)sulphamoyl, N-(methyl)sulphamoyl and N-ethyl)sulphamoyl. Examples of “N-(C₁₋₆alkyl)₂sulphamoyl” and

“N-4alkyl)₂sulphamoyl” are N,N-(dimethyl)sulphamoyl and N-(methyl)—N-(ethyl)sulphamoyl. Examples of “N-(C₁₋₆alkyl)carbamoyl” and “N-(C₁₋₄alkyl)carbamoyl” are methylaminocarbonyl and ethylaminocarbonyl. Examples of “N,N-(C₁₋₆alkyl)₂carbamoyl” and “N,N-(C₁₋₄alkyl)₂carbamoyl” are dimethylaminocarbonyl and methylethylaminocarbonyl. Examples of “C₁₋₆alkoxycarbonylamino” are ethoxycarbonylamino and t-butoxy-carbonylamino. Examples of “N′-(C₁₋₆alkyl)ureido” are N′-methylureido and N′-ethylureido. Examples of “N-(C₁₋₆alkyl)ureido are N-methylureido and N-ethylureido. Examples of “N′′,N′-(C₁₋₆alkyl)₂ureido are N′,N′-dimethylureido and N′-methyl—N′-ethylureido. Examples of “N′-(C₁₋₆alkyl)—N-(C₁₋₆alkyl)ureido are N′-methyl—N-methylureido and N′-propyl—N-methylureido. Examples of “N′,N′-(C₁₋₆alkyl)₂—N-(C₁₋₆alkyl)ureido are N′,N′-dimethyl—N-methylureido and N′-methyl—N′-ethyl—N-propylureido.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid.

In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl) amine.

A prodrug of any compound mentioned herein as an IBAT inhibitor or a compound for use in combination therewith is a drug which is broken down in the human or animal body to give the compound.

The compounds of the formula (I) may be administered in the form of a pro-drug which is broken down in the human or animal body to give a compound of the formula (I).

Examples of pro-drugs include in vivo hydrolysable esters and in vivo hydrolysable amides of a compound of the formula (I).

An in vivo hydrolysable ester of a compound of the formula (I) containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl;1,3-dioxolen-2-onylmethyl esters for example 5-methyl-1,3-dioxolen-2-onylmethyl; and C₁₋₆alkoxycarbonyloxyethyl esters for example 1-methoxy-carbonyloxyethyl and may be formed at any carboxy group in the compounds of this invention.

An in vivo hydrolysable ester of a compound of the formula (I) containing a hydroxy group includes inorganic esters such as phosphate esters and a-acyloxyalkyl ethers and related compounds which as a result of the in vivo hydrolysis of the ester breakdown to give the parent hydroxy group. Examples of a-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N(dialkylaminoethyl)—N-alkylcarbamoyl (to give carbamates), dialkylaminoacetyl and carboxyacetyl. Examples of substituents on benzoyl include morpholino and piperazino linked from a ring nitrogen atom via a methylene group to the 3-or 4-position of the benzoyl ring.

A suitable value for an in vivo hydrolysable amide of a compound of the formula (I) containing a carboxy group is, for example, a N—C₁₋₆alkyl or N,N-d1—C₁₋₆alkyl amide such as N-methyl, N-ethyl, N-propyl, N,N-dimethyl, N-ethyl—N-methyl or N,N-diethyl amide. It is also to be understood that certain compounds of the formula (I) can exist in solvated as well as unsolvated forms such as, for example, hydrated forms. It is to be understood that the invention encompasses all such solvated forms which possess IBAT inhibitory activity.

Preferred values of R¹, R², R³, R⁴, R⁵ and R⁶ are as follows. Such values may be used where appropriate with any of the definitions, claims or embodiments defined hereinbefore or hereinafter.

Preferably R¹ and R² are independently selected from C₁₋₄alkyl.

More preferably R¹ and R² are independently selected from ethyl or butyl.

More preferably R¹ and R² are independently selected from ethyl, propyl or butyl.

In one aspect of the invention particularly R¹ and R² are both butyl.

In a further aspect of the invention particularly R¹ and R² are both propyl.

In another aspect of the invention particularly one of R¹ and R² is ethyl and the other is butyl.

Preferably R^{x} and R^{Y} are independently selected from hydrogen or C₁₋₆alkyl. More preferably R^{x} and R^{Y} are both hydrogen.

Preferably R^{Z} is selected from halo, amino, C₁₋₆alkyl, C₁₋₆alkoxycarbonylamino or N′-(C₁₋₆alkyl)ureido.

More preferably R^{z} is selected from chloro, amino, t-butyl, t-butoxycarbonylamino or N′-(t-butyl)ureido.

Preferably v is 0 or 1.

In one aspect of the invention, more preferably v is 0.

In one aspect of the invention, more preferably v is 1.

In one aspect of the invention preferably R⁴ is a group of formula (IA) (as depicted above).

In another aspect of the invention preferably R⁵ is a group of formula (IA) (as depicted above).

Preferably R³ and R⁶ are hydrogen.

Preferably the other of R⁴ and R⁵ that is not the group of formula (IA) is selected from halo,

C₁₋₄alkoxy or C₁₋₄alkylS(O), wherein a is 0 to 2; wherein that R⁴ or R⁵ may be optionally substituted on carbon by one or more R¹⁶; wherein R¹⁶ is independently selected from hydroxy and N,N-(C₁₋₄alkyl)₂amino.

More preferably the other of R⁴ and R⁵ that is not the group of formula (IA) is selected from bromo, methoxy, isopropoxy, methylthio, ethylthio, isopropylthio or mesyl; wherein that R⁴ or R⁵ may be optionally substituted on carbon by one or more R¹⁶; wherein R¹⁶ is independently selected from hydroxy and N,N-dimethylamino.

Particularly the other of R⁴ and R⁵ that is not the group of formula (IA) is selected from bromo, methoxy, isopropoxy, methylthio, ethylthio, isopropylthio, 2-hydroxyethylthio, 2-(N,N-dimethylamino) ethylthio or mesyl.

More particularly the other of R⁴ and R⁵ that is not the group of formula (IA) is methylthio.

Preferably the other of R⁴ and R⁵ that is not the group of formula (IA) is selected from hydrogen, halo, C₁₋₄alkoxy or C₁₋₄alkylS(O)ₐ wherein a is 0 to 2; wherein that R⁴ or R⁵ may be optionally substituted on carbon by one or more R¹⁶; wherein R¹⁶ is independently selected from hydroxy, carboxy and N,N-(C₁₋₄alkyl)₂amino.

More preferably the other of R⁴ and R⁵ that is not the group of formula (IA) is selected from hydrogen, bromo, methoxy, isopropoxy, methylthio, ethylthio, isopropylthio or mesyl; wherein that R⁴ or R⁵ may be optionally substituted on carbon by one or more R¹⁶; wherein R¹⁶ is independently selected from hydroxy, carboxy and N,N-dimethylamino.

Particularly the other of R⁴ and R⁵ that is not the group of formula (IA) is selected from hydrogen, bromo, methoxy, isopropoxy, methylthio, carboxymethylthio, ethylthio, isopropylthio, 2-hydroxyethylthio, 2-(N,N-dimethylamino) ethylthio or mesyl. In another aspect of the invention, more preferably the other of R⁴ and R⁵ that is not the group of formula (IA) is selected from hydrogen, chloro, bromo, methoxy, isopropoxy, methylthio, ethylthio or isopropylthio; wherein that R⁴ or R⁵ may be optionally substituted on carbon by one or more R¹⁶; wherein R¹⁶ is independently selected from hydroxy, carboxy and N,N-dimethylamino.

In another aspect of the invention, particularly the other of R⁴ and R⁵ that is not the group of formula (IA) is selected from hydrogen, chloro, bromo, methoxy, isopropoxy, methylthio, carboxymethylthio, ethylthio, isopropylthio, 2-hydroxyethylthio or 2-(N,N-dimethylamino) ethylthio.

In another aspect of the invention, more particularly the other of R⁴ and R⁵ that is not the group of formula (IA) is bromo or chloro.

In another aspect of the invention, more particularly the other of R⁴ and R⁵ that is not the group of formula (IA) is methoxy.

In one aspect of the invention, preferably Ring A is aryl.

In another aspect of the invention, preferably Ring A is heteroaryl.

When Ring A is aryl, preferably Ring A is phenyl.

When Ring A is heteroaryl, preferably Ring A is thienyl or indolyl.

Preferably Ring A is aryl or heteroaryl; wherein Ring A is optionally substituted by one or more substituents selected from R¹⁷; wherein R¹⁷ is selected from halo, hydroxy or CI 4alkyl; wherein R¹⁷ may be optionally substituted on carbon by one or more R²¹; wherein R²¹ is selected from halo.

Preferably X is —O.

More preferably Ring A is phenyl, thienyl or indolyl; wherein Ring A is optionally substituted by one or more substituents selected from halo, hydroxy or trifluoromethyl.

Particularly Ring A is selected from phenyl, 4-hydroxyphenyl, thien-2-yl, 4-trifluoromethylphenyl, 3-hydroxyphenyl, 2-fluorophenyl, 2,3-dihydroxyphenyl or indol-3-yl.

More particularly Ring A is phenyl.

In another aspect of the invention, preferably Ring A is aryl or heteroaryl; wherein Ring A is optionally substituted by one or more substituents selected from R¹⁷; wherein R¹⁷ is selected from halo, hydroxy, C₁₋₄alkyl or C₁₋₄alkoxy; wherein R¹⁷ may be optionally substituted on carbon by one or more R²¹; wherein R²¹ is selected from halo.

In another aspect of the invention, more preferably Ring A is phenyl, thienyl or indolyl; wherein Ring A is optionally substituted by one or more substituents selected from halo, hydroxy, methoxy or trifluoromethyl.

In another aspect of the invention, particularly Ring A is selected from phenyl, 4-hydroxyphenyl, 4-methoxyphenyl, thien-2-yl, 4-trifluoromethylphenyl, 3-hydroxyphenyl, 2-fluorophenyl, 2,3-dihydroxyphenyl or indol-3-yl.

In a further aspect of the invention, particularly Ring A is selected from phenyl, 4-hydroxyphenyl, 4-methoxyphenyl, thien-2-yl, 4-trifluoromethylphenyl, 3-hydroxyphenyl, 2-fluorophenyl, 4-fluorophenyl, 2,3-dihydroxyphenyl or indol-3-yl.

Preferably R⁷ is hydrogen, C₁₋₄alkyl or carbocyclyl.

More preferably R⁷ is hydrogen, methyl or phenyl.

Particularly R⁷ is hydrogen.

In one aspect of the invention, preferably R⁸ is hydrogen.

In another aspect of the invention, preferably R⁸ is C₁₋₄alkyl.

In another aspect of the invention, more preferably R⁸ is hydrogen or methyl.

In one aspect of the invention, preferably R⁹ is hydrogen.

In another aspect of the invention, preferably R⁹ is C₁₋₄alkyl.

In another aspect of the invention, more preferably R⁹ is hydrogen or methyl.

Preferably R¹⁰ is hydrogen.

In one aspect of the invention, preferably R¹¹ is carboxy, sulpho, sulphino, phosphono, —P(O)(OR^{c})(OR^{d}), —P (O)(OH)(OR^{c}), —P(O)(OH)(R^{d}) or —P(O)(OR^{c}) (R^{d}) wherein R^{c} and R^{d} are independently selected from C₁₋₆alkyl.

In another aspect of the invention, preferably R¹¹ is a group of formula (IB) (as depicted above).

Preferably R¹¹ is carboxy, —P(O)(OH)(OR^{c}) or a group of formula (IB) (as depicted above).

More preferably R¹¹ is carboxy, —P(O)(OH)(OEt) or a group of formula (IB) (as depicted above).

In another aspect of the invention, preferably R¹¹ is carboxy, sulpho, —P(O)(OH)(OR^{c}) wherein R^{c} is selected from C₁₋₄alkyl or a group of formula (IB) (as depicted above).

Preferably Y is —NH— or —NHC (O)—.

More preferably Y is —NHC (O)—.

In one aspect of the invention, preferably R¹² is hydrogen.

In another aspect of the invention, preferably R¹² is C₁₋₄alkyl.

In another aspect of the invention, more preferably R¹² is hydrogen or methyl.

Preferably R¹³ is hydrogen, C₁₋₄alkyl or carbocyclyl; wherein R¹³ is optionally substituted by one or more substituents selected from R²⁰; wherein R²⁰ is hydroxy.

More preferably R¹³ is hydrogen, methyl or phenyl; wherein R¹³ is optionally substituted by one or more substituents selected from R²⁰; wherein R²⁰ is hydroxy.

Particularly R¹³ is hydrogen, hydroxymethyl or phenyl.

More particularly R¹³ is hydrogen or hydroxymethyl.

In another aspect of the invention, preferably R¹³ is hydrogen, C₁₋₄alkyl or carbocyclyl; wherein R¹³ is optionally substituted by one or more substituents selected from R²⁰; wherein R²⁰ is hydroxy, carboxy, carbocyclyl or amino; wherein R²⁰ may be optionally substituted on carbon by one or more R²²; R²² is hydroxy.

In another aspect of the invention, more preferably R¹³ is hydrogen, methyl, ethyl, butyl or phenyl; wherein R¹³ is optionally substituted by one or more substituents selected from R²⁰; wherein R²⁰ is hydroxy, carboxy, phenyl or amino; wherein R²⁰ may be optionally substituted on carbon by one or more R²²; R²² is hydroxy.

In another aspect of the invention, particularly R¹³ is hydrogen, hydroxymethyl, 4-aminobutyl, 2-carboxyethyl, 4-hydroxybenzyl or phenyl.

In a further aspect of the invention, preferably R¹³ is hydrogen, C₁₋₄alkyl or carbocyclyl; wherein R¹³ is optionally substituted by one or more substituents selected from R²⁰; wherein R²⁰ is hydroxy, carboxy, carbocyclyl, heterocyclyl or amino; wherein R²⁰ may be optionally substituted on carbon by one or more R²²; R²² is hydroxy.

In a further aspect of the invention, more preferably R¹³ is hydrogen, methyl, ethyl, butyl or phenyl; wherein R¹³ is optionally substituted by one or more substituents selected from R²⁰; wherein R²⁰ is hydroxy, carboxy, phenyl, imidazolyl or amino; wherein R²⁰ may be optionally substituted on carbon by one or more R²²; R²² is hydroxy.

In a further aspect of the invention, particularly R¹³ is hydrogen, hydroxymethyl, 4-aminobutyl, 2-carboxyethyl, 4-hydroxybenzyl, imidazol-5-ylmethyl or phenyl.

In another further aspect of the invention, preferably R¹³ is hydrogen, C₁₋₄alkyl, carbocyclyl or R²³; wherein R¹³ is optionally substituted by one or more substituents selected from R²⁰;

wherein R²⁰ is hydroxy, C₁₋₄alkylS (O) a wherein a is 0, C₁₋₄alkoxy, amino, carbocyclyl, heterocyclyl or mercapto; wherein R²⁰ may be independently optionally substituted on carbon by one or more R²²; R²² is selected from hydroxy; and R²³ is carboxy.

In another further aspect of the invention, more preferably R¹³ is hydrogen, methyl, ethyl, butyl or phenyl or R²³; wherein R¹³ is optionally substituted by one or more substituents selected from R²⁰; wherein R²⁰ is hydroxy, methylthio, methoxy, amino, imidazolyl or mercapto; wherein R²⁰ may be independently optionally substituted on carbon by one or more R²²; R²² is selected from hydroxy; and R²³ is carboxy.

In another further aspect of the invention, particularly R¹³ is hydrogen, carboxy, hydroxymethyl, mercaptomethyl, methoxymethyl, methylthiomethyl, 2-methylthioethyl, 4-aminobutyl, 4-hydroxybenzyl, imidazol-5-ylmethyl or phenyl.

In another aspect more particularly R¹³ is methylthiomethyl, methylsulphinylmethyl or methylsulphonylmethyl.

Preferably R¹⁴ is hydrogen.

In another aspect of the invention, preferably R14 is selected from hydrogen, C₁₋₄alkyl or carbocyclyl; wherein said C₁₋₄alkyl or carbocyclyl may be optionally substituted by one or more substituents selected from R²⁰; and R²⁰ is hydroxy.

In another aspect of the invention, more preferably R¹⁴ is selected from hydrogen, methyl or phenyl; wherein said methyl or phenyl may be optionally substituted by one or more substituents selected from R²⁰; and R²⁰ is hydroxy.

In another aspect of the invention, particularly R¹⁴ is hydrogen, phenyl or hydroxymethyl. Particularly R¹⁵ is carboxy or sulpho.

In one aspect of the invention, more particularly R¹⁵ is carboxy.

In another aspect of the invention, more particularly R¹⁵ is sulpho.

Preferably R¹⁵ is carboxy, sulpho,—P(O)(OR^{e}) (OR'), —P(O)(OH)(ORe), —P(O)(OH)(Re) or —P(O)(OR^{e})(R^{f}) wherein R^{e} and R^{f} are independently selected from C₁₋₄alkyl. More preferably R¹⁵ is carboxy, sulpho, —P(O)(OR^{e})(OR^{f}), —P(O)(OH)(OR^{e}), —P(O)(OH)(Re) or - P(O)(ORe)(R^{f}) wherein Re and R^{f} are independently selected from methyl or ethyl.

Preferably R¹⁵ is carboxy, sulpho, —P(O)(OEt)(OEt), —P(O)(OH)(OEt), —P(O)(OH)(Me) or —P(O)(OEt)(Me).

Preferably R¹⁵ is carboxy, sulpho, phosphono, —P(O)(OR^{e})(OR^{f}), —P(O)(OH)(OR^{e}), —P(O)(OH) (R^{e}) or —P(O)(ORe)(R^{f}) wherein Re and R^{f} are independently selected from C₁₋₄alkyl or R¹⁵ is a group of formula (IC) (as depicted above).

More preferably R¹⁵ is carboxy, sulpho, phosphono,—P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from methyl or ethyl or R¹⁵ is a group of formula (IC) (as depicted above).

Preferably R¹⁵ is carboxy, sulpho, phosphono, —P(O)(OEt)(OEt), —P (O)(Ot-Bu)(Ot-Bu), —P(O)(OH)(OEt), —P (O)(OH)(Me) or —P(O)(OEt)(Me) or R¹⁵ is a group of formula (IC) (as depicted above).

In one aspect of the invention, preferably R¹⁵ is carboxy.

In another aspect of the invention, preferably R¹⁵ is sulpho.

In another aspect of the invention, preferably R¹⁵ is —P(O)(OH)(OEt).

In another aspect of the invention, preferably R¹⁵ is —P(O)(OH)(Me).

In another aspect of the invention, preferably R¹⁵ is —P(O)(OEt)(Me).

In one aspect of the invention, preferably R²⁴ is hydrogen.

In another aspect of the invention, preferably R²⁴ is C₁₋₄alkyl.

Preferably R²⁵ is hydrogen.

Preferably R²⁶ is carboxy.

Preferably p is 1 or 2; wherein the values of R¹³ may be the same or different.

In one aspect of the invention, more preferably p is 1.

In another aspect of the invention, more preferably p is 2; wherein the values of R¹³ may be the same or different.

In a further aspect of the invention, more preferably p is 3; wherein the values of R¹³ may be the same or different.

In one aspect of the invention, preferably q is 0.

In a further aspect of the invention, preferably q is 1.

In one aspect of the invention, preferably r is 0.

In one aspect of the invention, more preferably r is 1.

In another aspect of the invention, more preferably r is 2; wherein the values of R¹⁴ may be the same or different.

In a further aspect of the invention, more preferably r is 3; wherein the values of R¹⁴ may be the same or different.

Preferably m is 0.

In another aspect of the invention, preferably m is 0 or 1.

Preferably n is 1.

In another aspect of the invention, preferably n is 1 or 2.

Preferably z is 1.

The group of formula (IA) wherein R⁷ is hydrogen, methyl or phenyl, n is 1, Ring A is phenyl, thienyl or indolyl; wherein Ring A is optionally substituted by one or more substituents selected from halo, hydroxy or trifluoromethyl, m is 0 and R⁹ is carboxy, —P(O)(OH)(OR^{c}) or a group of formula (IB).

The group of formula (IA) wherein: X is —O—.

Ring A is phenyl, thienyl or indolyl; wherein Ring A is optionally substituted by one or more substituents selected from halo, hydroxy, methoxy or trifluoromethyl;

R⁷ is hydrogen, methyl or phenyl;

R⁸ is hydrogen or methyl;

R⁹ is hydrogen or methyl;

R¹⁰ is hydrogen;

m is 0-2 wherein the values of R¹⁰ may be the same or different; and R¹¹ is carboxy, —P(O)(OH)(OEt) or a group of formula (IB) (as depicted in claim 1); The group of formula (IB) wherein R¹⁰ is hydrogen, hydroxymethyl or phenyl, p is 1 or 2; wherein the values of R¹⁰ may be the same or different and R¹¹ is carboxy or sulpho. The group of formula (IB) wherein:

R¹² is hydrogen or methyl;

R¹³ is hydrogen, methyl, ethyl, butyl or phenyl or R²³; wherein R¹³ is optionally substituted by one or more substituents selected from R²⁰; R²⁰ is hydroxy, methylthio, methoxy, amino, imidazolyl or mercapto; wherein R²⁰ may be independently optionally substituted on carbon by one or more hydroxy; R²³ is carboxy; Y is —NH—or —NHC (O)—; R¹⁴ is selected from hydrogen, methyl or phenyl; wherein said methyl or phenyl may be optionally substituted by one or more substituents selected from hydroxy; R¹⁵ is carboxy, sulpho, phosphono, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from methyl or ethyl or R¹⁵ is a group of formula (IC) (as depicted in claim 1);

p is 1-3 wherein the values of R¹³ may be the same or different;

q is 0-1; and

r is 0-3 wherein the values of R¹⁴ may be the same or different;

The group of formula (IC) wherein

R²⁴ is hydrogen;

R²⁵ is hydrogen;

R²⁶ is carboxy; and

z is 1;

or a pharmaceutically acceptable salt, solvate, solvate of such a salt or a prodrug thereof.

Therefore in a further aspect of the invention, there is provided a compound of formula (I) as depicted above wherein:

R¹ and R² are independently selected from ethyl or butyl;

R³ and R⁶ are hydrogen;

R⁴ is selected from halo, C₁₋₄alkoxy or C₁₋₄alkylS(O), wherein a is 0 to 2; wherein that R⁴ may be optionally substituted on carbon by one or more R¹⁶; wherein R¹⁶ is independently selected from hydroxy and N,N—(C₁₋₄alkyl)₂amino;

R⁵ is a group of formula (IA);

Ring A is aryl or heteroaryl; wherein Ring A is optionally substituted by one or more substituents selected from R¹⁷; wherein

R¹⁷ is selected from halo, hydroxy or C₁₋₄alkyl; wherein R¹⁷ may be optionally substituted on carbon by one or more R²¹; wherein

R²¹ is selected from halo;

R⁷ is hydrogen, C₁₋₄alkyl or carbocyclyl;

R¹¹ is carboxy, —P(O)(OH)(OR^{c}) or a group of formula (IB) (as depicted above);

R¹³ is hydrogen, C₁₋₄alkyl or carbocyclyl; wherein R¹³ is optionally substituted by one or more substituents selected from R²⁰; wherein

R²⁰ is hydroxy;

R¹⁵ is carboxy or sulpho;

p is 1 or 2; wherein the values of R¹³ may be the same or different;

m is 0; and

n is 1;

or a pharmaceutically acceptable salt, solvate, solvate of such a salt or a prodrug thereof.

Therefore in an additional aspect of the invention, there is provided a compound of formula (I) as depicted above wherein:

R¹ and R² are both butyl or one of R¹ and R² is ethyl and the other is butyl;

R⁴ is methylthio;

R⁵ is a group of formula (IA) (as depicted above);

R³ and R⁶ are hydrogen;

Ring A is phenyl;

R⁷ is hydrogen;

R¹¹ is a group of formula (IB) (as depicted above);

R¹³ is hydrogen or hydroxymethyl;

R¹⁵ is carboxy or sulpho;

p is 1 or 2; wherein the values of R¹³ may be the same or different;

m is 0;

n is 1;

or a pharmaceutically acceptable salt, solvate, solvate of such a salt or a prodrug thereof.

Therefore in an additional further aspect of the invention, there is provided a compound of formula (I) as depicted above wherein:

R¹ and R² are independently selected from ethyl or butyl;

R³ and R⁶ are hydrogen;

R⁴ is selected from halo, C₁₋₄alkoxy or C₁₋₄alkylS(O), wherein a is 0 to 2; wherein that R⁴ may be optionally substituted on carbon by one or more R¹⁶; wherein R¹⁶ is independently selected from hydroxy and N,N—(C₁₋₄alkyl)₂amino;

R⁵ is a group of formula (IA);

Ring A is aryl or heteroaryl; wherein Ring A is optionally substituted by one or more substituents selected from R¹⁷;

R⁷ is hydrogen, C₁₋₄alkyl or carbocyclyl;

R⁸ is hydrogen or methyl;

R⁹ is hydrogen or methyl;

R¹¹ is carboxy, —P(O)(OH)(OR^{c}) or a group of formula (IB) (as depicted above);

X is —NH—or —NHC(O)—;

R¹² is hydrogen or methyl;

R¹³ is hydrogen, C₁₋₄alkyl or carbocyclyl; wherein R¹³ is optionally substituted by one or more substituents selected from R²⁰;

R¹⁴ is hydrogen;

R¹⁵ is carboxy or sulpho;

R¹⁷ is selected from halo, hydroxy, C₁₋₄alkyl or C₁₋₄alkoxy; wherein R¹⁷ may be optionally substituted on carbon by one or more R²¹;

R²⁰ is hydroxy, carboxy, carbocyclyl or amino; wherein R²⁰ may be optionally substituted on carbon by one or more R²²;

R²¹ is selected from halo;

R²² is hydroxy;

p is 1-3; wherein the values of R¹³ may be the same or different.

q is 0-1;

r is 0-3; wherein the values of R¹⁴ may be the same or different; and wherein if q is 1, r is not 0;

m is 0-2; and

n is 1-3;

or a pharmaceutically acceptable salt, solvate, solvate of such a salt or a prodrug thereof.

Therefore in another additional further aspect of the invention, there is provided a compound of formula (I) as depicted above wherein:

R¹ and R² are independently selected from C₁₋₄alkyl;

R^{x} and R^{y} are both hydrogen;

R^{z} is selected from halo, amino, C₁₋₆alkyl, C₁₋₆alkoxycarbonylamino or N′—(C₁₋₆alkyl)ureido;

v is 0 or 1;

R³ and R⁶ are hydrogen;

one of R⁴ and R⁵ is a group of formula (IA) (as depicted above) and the other is selected from hydrogen, halo, C₁₋₄alkoxy or C₁₋₄alkylS(O)ₐ wherein a is 0 to 2; wherein that R⁴ or R⁵ may be optionally substituted on carbon by one or more R¹⁶; wherein R¹⁶ is independently selected from hydroxy, carboxy and N,N—(C₁₋₄alkyl)2amino;

X is —O—

R⁷ is hydrogen, methyl or phenyl;

R⁸ is hydrogen or methyl;

Ring A is aryl or heteroaryl; wherein Ring A is optionally substituted by one or more substituents selected from R¹⁷; wherein R¹⁷ is selected from halo, hydroxy, C₁₋₄alkyl or C₁₋₄alkoxy; wherein R¹⁷ may be optionally substituted on carbon by one or more R²¹; wherein

R²¹ is selected from halo;

R⁹ is hydrogen or methyl;

R¹⁰ is hydrogen;

R¹¹ is carboxy, —P(O)(OH)(OR^{c}) wherein R^{c} is selected from C₁₋₄alkyl or a group of formula (IB) (as depicted above);

R¹² is hydrogen or methyl;

Y is —NH—or —NHC(O)—;

R¹³ is hydrogen, C₁₋₄alkyl, carbocyclyl or R²³; wherein R¹³ is optionally substituted by one or more substituents selected from R²⁰; wherein R²⁰ is hydroxy, C₁₋₄alkylS(O), wherein a is 0, C₁₋₄alkoxy, amino, carbocyclyl, heterocyclyl or mercapto; wherein R²⁰ may be independently optionally substituted on carbon by one or more R²²; R²² is selected from hydroxy; and R²³ is carboxy;

R¹⁴ is selected from hydrogen, C₁₋₄alkyl or carbocyclyl; wherein said C₁₋₄alkyl or carbocyclyl may be optionally substituted by one or more substituents selected from R²⁰; and R²⁰ is hydroxy;

R¹⁵ is carboxy, sulpho, phosphono, —P(O)(OR^{e})(OR^{f}), —P(O)(OH)(ORe), —P(O)(OH)(Re) or —P(O)(OR^{e})(R^{f}) wherein R^{e} and R^{f} are independently selected from C₁₋₄alkyl or R¹⁵ is a group of formula (IC) (as depicted above);

R²⁴ is hydrogen;

R²⁵ is hydrogen;

R²⁶ is carboxy;

p is 1-3; wherein the values of R¹³ may be the same or different;

q is 0-1;

r is 0-3; wherein the values of R¹⁴ may be the same or different;

m is 0-2; wherein the values of R¹⁰ may be the same or different;

n is 1-2; wherein the values of R⁷ may be the same or different;

z is 0-1; wherein the values of R²⁵ may be the same or different;

or a pharmaceutically acceptable salt, solvate, solvate of such a salt or a prodrug thereof.

Specific examples of compounds of formula I are substances of formula II

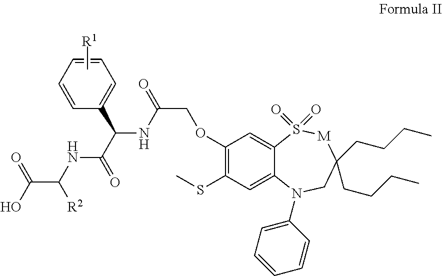

wherein

M is CH₂ or NH

R¹ is H or hydroxy

R² is H, CH₃, CH₂CH₃, CH₂CH₂CH₃, CH₂CH₂CH₂CH₃, CH(CH₃)₂, CH₂CH(CH₃)₂, CH(CH₃)CH₂CH₃, CH₂OH, CH₂OCH₃, CH(OH)CH₃, CH₂SCH₃, CH₂CH₂SCH₃.

Examples of useful substances according to the invention are:

1,1-Dioxo-3,3-dibutyl-5-phenyl-7-methylthio-8-(N-{(R)-α-[N-(carboxymethyl)carbamoyl]benzyl} carbamoylmethoxy)-2,3,4,5-tetrahydro-1,2,5-benzothiadiazepine,

1,1-Dioxo-3,3-dibutyl-5-phenyl-7-methylthio-8-(N-{(R)-α-[N′-((S)-1-carboxyethyl) carbamoyl]benzyl} carbamoylmethoxy)-2, 3,4, 5-tetrahydro-1, 5-benzothiazepine,

1,1-Dioxo-3,3-dibutyl-5-phenyl-7-methylthio-8-(N-{(R)-α-[N-((S)-1-carboxypropyl) carbamoyl]benzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α-[N-((R)-1-carboxy-2-methylthioethyl)carbamoyl]benzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α—N-((R)-1-carboxy-2-methylthio-ethyl)carbamoyl]-4-hydroxybenzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α—N-((S)-1-carboxy-2-methylpropylcarbamoyl]benzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α—N-((S)-1-carboxy-2-(R)-hydroxypropyl)carbamoyl]-4-hydroxybenzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α-[N-((S)-1-carboxybutyl) carbamoyl]-4-hydroxybenzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α-[N-((S)-1-carboxyethyl) carbamoyl]benzyl}carbamoylmethoxy)-2,3,4,5-tetrahydro-1,2,5-benzothiadiazepine,

1,1-Dioxo-3, 3-dibutyl-5-phenyl-7-methylthio-8-(N-{(R)-α-[N′-((S)-1-carboxypropyl) carbamoyl]-4-hydroxybenzyl} carbamoylmethoxy) -2, 3,4, 5-tetrahydro-1, 5-benzothiazepine,

1,1-Dioxo-3,3-dibutyl-5-phenyl-7-methylthio-8-(N-{(R)-α—N-((S)-1-carboxyethyl)carbamoyl]-4-hydroxybenzyl}carbamoylmethoxy)-2,3,4,5-tetrahydro-1,2,5-benzothiadiazepine,

1,1-Dioxo-3,3-dibutyl-5-phenyl-7-methylthio-8-(N-{(R)-α—N-((S)-1-carboxy-2-methylpropyl)carbamoyl]-4-hydroxybenzyl}carbamoylmethoxy)-2,3,4,5-tetrahydro-1,2,5-benzothiadiazepine and

1,1-Dioxo-3, 3-dibutyl-5-phenyl-7-methylthio-8-(N-{ (R)-1′-phenyl-1′-[N′-(carboxymethyl) carbamoyl] methyl} carbamoylmethoxy)-2, 3,4, 5-tetrahydro-1, 5-benzothiazepine.

In another aspect of the invention, preferred compounds of the invention are any one of the examples or a pharmaceutically acceptable salt, solvate, solvate of such a salt or a prodrug thereof.

Some compounds of the formula (I) may have chiral centres and/or geometric isomeric centres (E-and Z-isomers), and it is to be understood that the invention encompasses all such optical, diastereoisomers and geometric isomers that possess IBAT inhibitory activity.

The invention relates to any and all tautomeric forms of the compounds of the formula (1) that possess IBAT inhibitory activity.

The invention also relates all possible isomers of the compounds of the invention such as, optical and/or geometrical, pure or as a mixture, in all proportions, of the said compounds of formulas I and II and those specifically mentioned and the possible tautomeric forms In certain embodiments, compounds described herein have one or more chiral centers. As 4such, all stereoisomers are envisioned herein. In various embodiments, compounds described herein are present in optically active or racemic forms. It is to be understood that the compounds of the present invention encompasses racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments mixtures of one or more isomer is utilized as the therapeutic compound described herein. In certain embodiments, compounds described herein contains one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

In another aspect of the invention, preferred compounds of the invention are any one of the Examples or a pharmaceutically acceptable salt, solvate, solvate of such a salt or a prodrug thereof.

The invention further regards a composition comprising a compound according to the invention for use in prophylaxis and treatment of metabolic syndrome, obesity, disorders of fatty acid metabolism, glucose utilization disorders, disorders in which insulin resistance is involved, diabetes mellitus, type 1 and type 2 diabetes.

It also relates to the use of a substance or a composition according to the invention for the preparation of a medicine or a pharmaceutical composition for the treatment of metabolic syndrome, obesity, disorders of fatty acid metabolism, glucose utilization disorders, disorders in which insulin resistance is involved, diabetes mellitus, type 1 and type 2 diabetes.

According to the invention the IBAT inhibitors with formula I and II above may be combined with least one other active substance. The active substance may be another substance with IBAT inhibitory effect.

Combination Therapy with Other Active Substances

In certain instances, provided herein are combination compositions and/or therapies comprising any compound described herein and an other active substance, which may be a L-cell endocrine peptide enhancer.

In one embodiment, the L-cell endocrine peptide enhancer is a PYY enhancer. Enhanced secretion of PYY may provide a reduction of hunger. The L-cell endocrine peptide enhancer may be an oxyntomodulin enhancer. In some instances, the enhanced secretion of oxyntomodulin inhibits meal-stimulated gastric secretion.

Another useful L-cell endocrine peptide enhancer is a GLP-1 enhancer. Examples of GLP-1 enhancers are GLP-1, a GLP-1 secretion enhancer, a GLP-1 degradation inhibitor and the like, or a combination thereof. In certain instances, enhanced GLP-1 concentration provides a reduction in food intake and/or a reduction in gastric emptying in human subjects.

The L-cell endocrine peptide enhancer may also be a GLP-2 enhancer, such as a GLP-2, a GLP-2 secretion enhancer, a GLP-2 degradation inhibitor, etc. or a combination thereof. According to one embodiment, enhanced GLP-2 secretion inhibits gastric emptying and reduces intestinal permeability and/or the enhanced GLP-2 secretion inhibits gastric acid secretion. Enhanced GLP-2 secretion may also reduce or prevent inflammation in the gastrointestinal tract (gastrointestinal enteritis) and/or regenerate and/or heal injury to gastrointestinal tissues (e.g., radiation enteritis).

In some instances, the other active substance modulates bile acid receptors in the gastrointestinal lumen and or other organs. In some embodiments, the other active substance substamtially or partially agonizes bile acid receptors (e.g., TGR5 receptors or Farnesoid-X receptors) in the gastrointestinal tract. The other active substance In some embodiments, the additional therapeutic agent may be a bile acid analogue. In certain instances the additional therapeutic agent is a TGR5 agonist. Administration of a TGR5 agonist in combination with any of the compounds described herein may enhance the secretion of enteroendocrine peptides from L-cells. TGR5 modulators (e.g., agonists) include, and are not limited to, the compounds described in, WO 2008/091540, WO 2008/067219 and U.S. Appl. No. 2008/0221161.

In some embodiments, other active substance is a biguanide. A biguanide may reduce blood and/or plasma glucose levels. Examples of biguanides include and are not limited to metformin, phenformin, buformin, proguanil or the like.

In some embodiments, the other active substances are selected from enteroendocrine peptides. They may reverse insulin resistance and lower blood and/or plasma glucose levels. Examples of enteroendocrine peptides include but are not limited to GLP-1 or GLP-1 analogues such as Taspoglutide.RTM. (Ipsen) or the like.

In another embodiment, the other active substance is a thiazolidinedione. Thiazolidinediones may reverse insulin resistance and lower blood and/or plasma glucose levels. Examples of thiazolidinediones include and are not limited to Rosiglitazone (Avandia), Pioglitazone (Actos), Troglitazone (Rezulin), MCC-555, rivoglitazone, ciglitazone or the like.

In some embodiments, the additional therapeutic agent is an incretin mimetic, which could mimic augments pancreas response to ingestion of food, in some instances, administration of an incretin mimetic in combination with any of the compounds described herein lowers blood and/or plasma glucose levels. Examples of incretin mimetics include but are not limited to exenatide (Byetta™).

One currently used therapy for the treatment of diabetes is a subcutaneous injection of exenatide (Byetta™). In some embodiments, an oral combination of an IBAT inhibitor and a DPP-IV inhibitor is equally or more effective than an injection of exenatide in reducing plasma glucose levels. In some embodiments, an oral combination of an IBAT inhibitor and a DPP-IV inhibitor reduces or eliminates discomfort associated with injections of glucose-lowering medications.

According to the invention an IBAT inhibitor may be used together with a DPP-IV Inhibitor. In some embodiments, the other active substance inhibits degradation of L-cell enteroendocrine peptides. In certain embodiments, the other active substance is a DPP-IV inhibitor. Administration of an IBAT inhibitor to an individual in need thereof may enhance the secretion of GLP-1. Administration of a DPP-IV inhibitor in combination with the IBAT inhibitor may reduce or inhibit degradation of GLP-1 thereby prolonging the therapeutic benefit of enhanced levels of GLP-1. In some instances, administration of an IBAT inhibitor reduces weight of an individual. Thus, administration of an IBAT inhibitor in combination with a DPP-IV inhibitor may reduce weight of an individual.

DPP-IV inhibitors may be selected from (2S)-1-{2-[(3-hydroxy-1-adamantyl)amino]acetyl}pyrrolidine-2-carbonitrile (vildagliptin), (3R)-3-amino-1-[9-(trifluoromethyl)-1,4,7,8-tetrazabicyclo[4.3.0]nona-6,8- -dien-4-yl]-4-(2,4,5-trifluorophenyl)butan-1-one (sitagliptin), (1S,3S,5S)-2-[(2S)-2-amino-2-(3-hydroxy-1-adamantypacetyl]-2-azabicyclo[3.1.0]hexane-3-carbonitrile (saxagliptin), and 2-({6-[(3R)-3-aminopiperidin-1-yl]-3-methyl-2,4-dioxo-3,4-dihydropyrimidi-n-1(2H)-yl}methyl)benzonitrile (alogliptin).

Another therapy that is current standard of care for the treatment of diabetes is a combination of metformin and sitagliptin (Janumet™). At doses of 0,3-300 mg/kg metformin in combination with 30 mg/kg of sitagliption, induce reduction in plasma glucose concentrations from 3 hours till about 6 hours post-dose. In some embodiments, a combination of an IBAT inhibitor and sitagliptin maintains reduced plasma glucose concentrations for a longer duration of time compared to a combination of metformin and sitagliptin. In some instances IBAT inhibitor therapy eliminates side effects associated with metformin therapy and/or DPP-IV inhibitor therapy.

In some embodiments of any of the methods described herein, administration of an ASBT inhibitor described herein in combination with a DPP-IV inhibitor increases the level of GLP-1 in the blood and/or plasma of an individual by from about 1.5 times to about 30 times compared to the level of GLP-1 in the blood and/or plasma of the individual prior to administration of the IBAT inhibitor in combination with the DPP-IV inhibitor.

In some instances, an increase in GLP-1 level of from about 2 times to about 3 times following the administration of an ASBT inhibitor described herein in combination with a DPP-IV inhibitor compared to the level of GLP-1 in the blood and/or plasma of the individual prior to administration of the IBAT inhibitor in combination with the DPP-IV inhibitor is associated with an anti-diabetic effect and/or with reduction in food intake and/or induction of satiety and/or weight loss.

In some embodiments of any of the methods described herein, administration of an IBAT inhibitor in combination with a DPP-IV inhibitor reduces blood and/or plasma sugar levels for a longer period of time (e.g., at least 24 hours) compared to reduction in blood and/or plasma sugar levels upon administration of metformin in combination with a DPP-IV inhibitor.

In some embodiments of any of the methods described herein, administration of a single dose of an IBAT inhibitor in combination with a DPP-IV inhibitor sustains reduced blood and/or plasma sugar levels for at least 6 hours, at least 12 hours, at least 14 hours, at least 16 hours, at least 18 hours, at least 20 hours, at least 24 hours, at least 30 hours, at least 36 hours or at least 48 hours compared to reduction in blood and/or plasma sugar levels upon administration of a single dose of metformin in combination with a DPP-IV inhibitor.

According to one embodiment, administration of an IBAT inhibitor in combination with a DPP-IV inhibitor reduces blood and/or plasma sugar levels by at least 20%, at least 30%, at least 40%, at least 50% at least 60%, at least 70% or at least 80% compared to blood and/or plasma sugar levels prior to administration of the IBAT linhibitor in combination with a DPP-IV inhibitor.

In some embodiments of any of the methods described herein, administration of an IBAT INHIBITOR in combination with a DPP-IV inhibitor reduces blood and/or plasma sugar levels by at least 20%, e.g. at least 30%, such as at least 40%, e.g. at least 50% compared to blood and/or plasma sugar levels prior to administration of the IBAT inhibitor in combination with a DPP-IV inhibitor.

According to one embodiment of the invention, administration of an IBAT inhibitor in combination with a DPP-IV inhibitor results in higher levels of GLP-1 in blood and/or plasma of an individual compared to levels of GLP-1 in blood and/or plasma of a normal individual. In some embodiments of any of the methods described herein, administration of an IBAT inhibitor in combination with a DPP-IV inhibitor results in higher levels of GLP-1 in blood and/or plasma of an individual compared to levels of GLP-1 in blood and/or plasma of an individual undergoing therapy with metformin and/or a DPP-IV inhibitor.

In some embodiments, an IBAT inhibitor is administered in combination with a DPP-IV inhibitor and/or a biliary shunt. Biliary shunts may be selected from the shunts described in WO 2007/0050628, which is incorporated herein by reference. A biliary shunt may move bile acid to the distal ileum and/or the rectum and/or the colon thereby increasing the concentration of bile acids in the vicinity of L-cells present in the distal portion of the gastrointestinal tract. Such an increase in the concentration of bile acids in the vicinity of L-cells increases in some instances the secretion of GLP-1 from L-cells thereby inducing satiey and/or reduction in hunger and/or weight loss and/or reduction in plasma glucose levels or any combination thereof.

The other active substance and the IBAT inhibitor are used such that the combination is present in a therapeutically effective amount. For example an IBAT inhibitor and the other active substance (e.g., a DPP-IV inhibitor) are each is used in a therapeutically effective amount. When an additive or synergistic effect is present, they can each be used in a subclinical therapeutically effective amount.

In some embodiments, the use of a combination of an IBAT inhibitor and any other active ingredient as described herein encompasses combinations where the IBAT inhibitor or the other active ingredient is present in a therapeutically effective amount, and the other is present in a subclinical therapeutically effective amount, provided that the combined use is therapeutically effective owing to their additive or synergistic effects. As used herein, the term “additive effect” describes the combined effect of two (or more) pharmaceutically active agents that is equal to the sum of the effect of each agent given alone. A synergistic effect is one in which the combined effect of two (or more) pharmaceutically active agents is greater than the sum of the effect of each agent given alone. Any suitable combination of an ASBTI with one or more of the aforementioned other active ingredients and optionally with one or more other pharmacologically active substances is contemplated as being within the scope of the methods described herein.

Methods for experimentally determining therapeutically-effective dosages of drugs and other agents for use in combination treatment regimens are described in the literature.

The compounds may be administered concurrently e.g., simultaneously, essentially simultaneously or within the same treatment protocol or sequentially, depending upon the nature of the disease, disorder, or condition, the condition of the individual, and the actual choice of compounds used.

The multiple therapeutic agents are optionally administered in any order or simultaneously. If simultaneously, the multiple therapeutic agents are optionally provided in a single, unified form or in multiple forms, e.g. as a single pill or as two separate pills. In certain instances, one of the therapeutic agents is optionally given in multiple doses. In other instances, both are optionally given as multiple doses. If not simultaneous, the timing between the multiple doses may be, e.g., from more than a couple of days to less than four weeks. In addition, the combination methods, compositions and formulations are not to be limited to the use of only two agents. The use of multiple therapeutic combinations is also envisioned including two or more of the active substances described herein.

The active substances in a combination therapy described herein may be provided in a combined dosage form or in separate dosage forms intended for substantially simultaneous administration. In some embodiments, the active compounds are administered sequentially, with either therapeutic compound being administered by a regimen using a two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In certain embodiments, a dosage regimen to treat, prevent, or ameliorate the condition(s), is modified depending on e.g. the disorder from which the subject suffers, as well as the age, weight, sex, diet, and medical condition of the subject. Thus, in various embodiments, the dosage regimen actually employed may differ from the dosage regimens set forth herein. In certain embodiments, IBAT inhibitor compounds described herein are combined with or one or more of: insulin, insulin-mimetics, incretin mimetics, GLP-1 or analogues thereof,

GLP-2 or analogues thereof, oxyntomodulin, PYY, DPP-IV inhibitors, or TGR5 modulators in any combination.

The invention also regards IBAT inhibitor compounds described herein in combination with at least one bile acid binder e.g. a resin such as cholestyramine, cholestipol and colesevelam.

Bile acid binders (bile acid sequestrants, resins)

The following bile acid binders may be used according to the invention.

Cholestyramine a hydrophilic polyacrylic quaternary ammonium anion exchange resin, which is known to be effective in reducing blood cholesterol levels. Cholestyramine, and various compositions including cholestyramine, are described, for example, in British Pat Nos. 929,391 and 1,286, 949; and U.S. Pat. Nos. 3,383,281; 3,308,020; 3,769,399; 3,846,541; 3,974,272; 4,172,120; 4,252,790; 4,340,585; 4,814,354; 4,874,744; 4,895,723; 5,695,749; and 6,066,336. Cholestyramine is commercially available from Novopharm, USA Inc (Questrans Light), Upsher-Smith (PREVALITE (D) and Apothecon. As used herein, “cholestyramine” includes any such composition comprising cholestyramine, or pharmaceutically acceptable salts thereof. These are also called Questrans™ Questran Light Questrans Light (cholestyramine) is a non-absorbable anion binding resin FDA approved for the treatment of hypercholesterolemia.

An amine polymer having a first substituent, bound to a first amine of the amine polymer, that includes a hydrophobic aliphatic moiety, and a second substituent, bound to a second amine of the amine polymer that includes an aliphatic quaternary amine-containing moiety as described in U.S. Pat. No. 5,693,675 and 5,607,669.

The salt of an alkylated and crosslinked polymer comprising the reaction product of: (a) one or more crosslinked polymers, or salts and copolymers thereof having a repeat unit selected from the group consisting of: (NR—CH₂CH₂)n (2) and (NR—CH₂CH₂—NR—CH₂CH₂—NR—CH₂CHOH—CH₂)n (3) where n is a positive integer and each R, independently, is H or a C1—C8 alkyl group; (b) at least one aliphatic alkylating agent, said reaction product characterized in that: (i) at least some of the nitrogen atoms in said repeat units unreacted with said alkylating agent; (ii) less than 10 mol percent of the nitrogen atoms in said repeat units reacting with said alkylating agent forming quaternary ammonium units; and (iii) a fixed positive charge and one or more counterions, such as Colesevelam and colesevelam hydrochlorid.

Suitable bile acid binders for such a combination therapy are resins, such as cholestyramine and cholestipol. One advantage is that the dose of bile acid binder might be kept lower than the therapeutic dose for treatment of cholesterolaemia in single treatment comprising solely a bile acid binder. By a low dose of bile acid binder any possible side effects caused by poor tolerance of the patient to the therapeutic dose could also be avoided.

Another useful bile acid binder is a water insoluble non-toxic polymeric amine having a molecular weight in excess of 3,000, having the property of binding at least 30% of the available glycocholic acid within 5 minutes when exposed to anaqueous solution of an equal weight of said acid, having a polymer skeleton inert to digestive enzymes, and having a water content greater than 65% after equilibration with air at 100% relative humidity, e,g, cholestipol described in U.S. Pat. No. 3,383,281.

In a further aspect of the invention a suitable bile acid binder is one of cholestyramine, cholestipol or colesevelam.

A preferred aspect of the present invention is the use of colesevelam as the bile acid binder.

According to an additional further aspect of the present invention there is provided a combination treatment comprising the administration of an effective amount of a IBAT inhibitor compound or a pharmaceutically acceptable salt, solvate, solvate of such a salt or a prodrug thereof, and a bile acid binder, wherein the formulation is designed to deliver the bile acid binder in the colon, with the simultaneous, sequential or separate administration one or more of the following agents selected from:

The compositions of the invention may further comprise statins e-g- an HMG Co-A reductase inhibitor, or a pharmaceutically acceptable salt, solvate, solvate of such a salt or a prodrug thereof, in association with a pharmaceutically acceptable diluent or carrier.

According to one embodiment the invention relates to a combined oral pharmaceutical formulation comprising an IBAT inhibitor compound or a pharmaceutically acceptable salt, solvate, solvate of such a salt or a prodrug thereof and/or a bile acid binder or a pharmaceutically acceptable salt, solvate, solvate of such a salt or a prodrug thereof, wherein the formulation is designed to deliver the bile acid binder in the colon and the IBAT inhibitor in the small intestine and wherein the combined formulation is intended for administration of the IBAT inhibitor and the bile acid binder simultaneously, separately or sequentially.

The IBAT inhibitor may be administrated once a day and the acid inhibitor one, two or three times a day. In one embodiment the IBAT inhibitor and the bile acid binder are administrated together one, two or three times a day.

In another embodiment the acid binder is formulated in separate formulation with the IBAT inhibitor formulation releasing the drug immediately or delayed in the distal jejunum or the proximal ileum and the bile acid binder formulation releasing the drug in the colon.

In still another embodioment, the formulation has a core comprising the bile acid binder formulated for release in the colon surrounded by an outer layer comprising IBAT inhibitor and formulated for immediate release or for delayed release in the distal jejunum or the proximal ileum.

Statins

In another aspect of the invention, an IBAT inhibitor compound e.g. a compound of formula (I) or (II) or a pharmaceutically acceptable salt, solvate, solvate of such a salt or a prodrug thereof, may be administered in association with an HMG Co-A reductase inhibitor, or pharmaceutically acceptable salts, solvates, solvates of such salts or prodrugs thereof. Suitable HMG Co-A reductase inhibitors, pharmaceutically acceptable salts, solvates, solvates of such salts or prodrugs thereof are statins well known in the art. Particular statins are fluvastatin, lovastatin, pravastatin, simvastatin, atorvastatin, cerivastatin, bervastatin, dalvastatin, mevastatin and (E)-7-[4-(4-fluorophenyl)-6-isopropyl-2-[methyl (methylsulphonyl) amino] pyrimidin-5-yl] (3R, 5S)-3,5-dihydroxyhept-6-enoic acid (rosuvastatin), or a pharmaceutically acceptable salt, solvate, solvate of such a salt or a prodrug thereof. A particular statin is atorvastatin, or a pharmaceutically acceptable salt, solvate, solvate of such a salt or a prodrug thereof. A more particular statin is atorvastatin calcium salt. A further particular statin is (E)-7-[4-(4-fluorophenyl)-6-isopropyl-2-[methyl (methylsulphonyl) amino] pyrimidin-5-yl] (3R, 5S)-3,5-dihydroxyhept-6-enoic acid (rosuvastatin), or a pharmaceutically acceptable salt, solvate, solvate of such a salt or a prodrug thereof. Other particular statin are rosuvastatin calcium salt and pitavastatin.

In an additional aspect of the invention, the compound of formula (I), or a pharmaceutically acceptable salt, solvate, solvate of such a salt or a prodrug thereof may be administered in association with an HMG Co-A reductase inhibitor, or a pharmaceutically acceptable salt, solvate, solvate of such a salt or a prodrug thereof, and/or a bile acid binder thereby avoiding a possible risk of excess of bile acids in colon caused by the inhibition of the ileal bile acid transport system. An excess of bile acids in the visceral contents may cause diarrhoea. Thus, the present invention also provides a treatment of a possible side effect such as diarrhoea in patients during therapy comprising the compound of formula (I), or a pharmaceutically acceptable salt, solvate, solvate of such a salt or a prodrug thereof.

An HMG CoA-reductase inhibitor, or a pharmaceutically acceptable salt, solvate, solvate of such a salt or a prodrug thereof will by its action decrease the endogenous cholesterol available for the bile acid synthesis and have an additive effect in combination with the compound of formula (I), or a pharmaceutically acceptable salt, solvate, solvate of such a salt or a prodrug thereof on lipid lowering.

A CETP (cholesteryl ester transfer protein) inhibitor, for example those referenced and described in WO 00/38725 page 7 line 22-page 10, line 17 which are incorporated herein by reference.

A cholesterol absorption antagonist for example azetidinones such as SCH 58235 and those described in U.S. pat. No. 5,767,115 which are incorporated herein by reference;

MTP (microsomal transfer protein) inhibitor for example those described in Science, 282,751-54,1998 which are incorporated herein by reference;

A fibric acid derivative; for example clofibrate, gemfibrozil, fenofibrate, ciprofibrate and bezafibrate;

A nicotinic acid derivative, for example, nicotinic acid (niacin), acipimox and niceritrol;

A phytosterol compound for example stanols;

Probucol ;

An anti-obesity compound for example orlistat (EP 129,748) and sibutramine (GB 2,184,122 and U.S. Pat. No. 4,929,629);

An antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, a diuretic or a vasodilator;

Insulin;

Sulphonylureas including glibenclamide and/or tolbutamide.

Acarbose;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

ACE inhibitors

Particular ACE inhibitors or pharmaceutically acceptable salts, solvates, solvate of such salts or a prodrugs thereof, including active metabolites, which can be used in combination with a compound of formula (I) include but are not limited to, the following compounds: alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. Preferred ACE inhibitors for use in the present invention are ramipril, ramiprilat, lisinopril, enalapril and enalaprilat. More preferred ACE inhibitors for uses in the present invention are ramipril and ramiprilat.

Angiotensin II antagonists

Preferred angiotensin II antagonists, pharmaceutically acceptable salts, solvates, solvate of such salts or a prodrugs thereof for use in combination with a compound of formula (I) include, but are not limited to, compounds: candesartan, candesartan cilexetil, losartan, valsartan, irbesartan, tasosartan, telmisartan and eprosartan. Particularly preferred angiotensin II antagonists or pharmaceutically acceptable derivatives thereof for use in the present invention are candesartan and candesartan cilexetil.

PPAR alpha and/or gamma and/or delta agonists or a pharmaceutically acceptable salt thereof

In another aspect of the invention, the IBAT inhibitor compound, or a pharmaceutically acceptable salt, solvate, solvate of such a salt or a prodrug thereof, may be administered in association with a PPAR alpha and/or gamma and/or delta agonist, or pharmaceutically acceptable salts, solvates, solvates of such salts or prodrugs thereof. Suitable PPAR alpha and/or gamma and/or delta agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, J Med Chem, 1996, 39,665, Expert Opinion on Therapeutic

Patents, 10 (5), 623-634 (in particular the compounds described in the patent applications listed on page 634) and J Med Chem, 2000,43,527, which are all incorporated herein by reference. Particularly a PPAR alpha and/or gamma agonist refers to WY-14643, clofibrate, fenofibrate, bezafibrate, GW 9578, troglitazone, pioglitazone, rosiglitazone, eglitazone, proglitazone, BRL-49634, KRP-297, JTT-501, SB 213068, GW 1929, GW 7845, GW 0207, L-796449, L-165041 and GW 2433.

Particularly a PPAR alpha and/or gamma agonist refers to (S)-2-ethoxy-3-[4-(2-{4-methanesulphonyloxyphenyl} ethoxy) phenyl] propanoic acid and pharmaceutically acceptable salts thereof.

Antidiabetics, hypoglycemic active ingredients, cholesterol absorption inhibitors, PPAR delta agonists, fibrates, MTP inhibitors, bile acid absorption inhibitors, polymeric bile acid adsorbents, LDL receptor inducers, ACAT inhibitors, antioxidants, lipoprotein lipase inhibitors, ATP-citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, alpha-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose-1,6-bisphosphatase, modulators of glucose transporter 4, inhibitors of glutamine-fructose-6-phosphate amidotransferase, inhibitors of dipeptidylpeptidase IV, inhibitors of 11-beta-hydroxysteroid dehydrogenase 1, inhibitors of protein tyrosine phosphatase 1 B, modulators of the sodium-dependent glucose transporter 1 or 2, modulators of GPR40, inhibitors of hormone-sensitive lipase, inhibitors of acetyl—CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY agonists, MC4 agonists, orexin agonists, H3 agonists, TNF agonists, CRF agonists, CRF BP antagonists, urocortin agonists, beta 3 agonists, CB1 receptor antagonists, MSH (melanocyte-stimulating hormone) agonists, CCK agonists, serotonin reuptake inhibitors, mixed serotoninergic and noradrenergic compounds, 5HT agonists, bombesin agonists, galanin antagonists, growth hormones, growth hormone-releasing compounds, TRH agonists, uncoupling protein 2 or 3 modulators, leptin agonists, DA agonists (bromocriptine, Doprexin), lipase/amylase inhibitors, PPAR modulators, RXR modulators or TR-beta-agonists or amphetamines.

Examples of PPAR delta agonists are GW-501516 (501516, GSK-516, GW-516, GW-1516;a peroxisome proliferator-activated receptor (PPAR)-delta agonist, and several other compounds developed from GW-501516, including GI-262570, GW-0072, GW-7845 and GW-7647.

According to one embodiment the IBAT inhibitor may be combined with one or more of

Atreleuton(5-LO) Eprotirome (THR-Beta), Losmapimod (p38MAPK), Ezetimibe (SCH58235) (NPC1L1) Bezafibrate, Fenofibrate, Varespladib, (sPLA2), Darapladib, (LpPLA2), Lomitapide, Implitapide, Rosiglitazone, Dalcetrapib, Anacetrapib, Lorcaserin, Dapagliflozin, Canagliflozin, Sergliflozin, ASP-1941, Orlistat, Exenatide, Liraglutide, Taspoglutide, Tulaglutide, Pramlintide, Lixisenatide, Albiglutide, Pioglitazone, Sodelglitazar, Netoglitazone,

Indeglitazar, Naveglitazar, Lobeglitazone, Aleglitazar, Bromocriptine, Tesofensine, Monoamine, Alogliptin, Vildagliptin, Saxagliptin, Sitagliptin, Denagliptin, Gemigliptin, Linagliptin, Dutogliptin, Teneligliptin, LC-150444, Laropiprant extended release niacin, Simvastatin ezetimibe, Rosuvastatin fenofibrate, Rosuvastatin ezetimibe and Atorvastatin ezetimibe.

Combinations with Tredaptive, Vytorin and Certriad may be used.

According to one embodiment the IBAT inhibitor may be combined with one or more of any of the above mentioned other compounds.

According to one embodiment the IBAT inhibitors of the present invention are combined with at least one other active substance selected from dipeptidyl peptidase-IV-inhibitors, PPAR y agonists, statins and bile acid binders in any combination.

According to one embodiment the IBAT inhibitors of the present invention are combined with at least one DPPIV, at least one PPAR y agonist, such as Sitagliptin and Pioglitazon.

According to one other embodiment the IBAT inhibitors of the present invention are combined with at least one DPPIV and at least one statin e.g. Sitagliptin and Simvastatin

According to one embodiment the at least one other substance may be chosen from dipeptidyl peptidase-IV inhibitors e.g. biguanides such as sitagliptin and; an incretin mimetic, a thiazolidinone, GLP-1 or an analogue thereof, and a TGR5 agonist. The at least one other substance with IBAT inhibitory effect may be chosen from metformin and non-absorbable sodium dependent bile transport inhibitors e.g. 1-[4-[4-R4R,5R)-3,3-dibutyl-7-(dimethylamino)-2,3,4,5-tetrahydro-4-hydroxy-1,1-dioxido-1-benzothiepin-5-yl]phenoxy]butyp-aza-1-azoniabicyclo[2.2.2]octane methane sulfonate.

Use of the substances and combinations of the inventio reduces or inhibits recycling of bile acid salts in the gastrointestinal tract. The bile transport inhibitors may be non-systemic and systemic compounds. They may enhance L-cell secretion of enteroendocrine peptides. In certain instances, increased L-cell secretion of enteroendocrine peptides is associated with induction of satiety and/or reduction of food intake and subsequent weight loss. In some embodiments, increased L-cell secretion of enteroendocrine peptides is associated with a reduction in blood and/or plasma glucose levels in a hyperglycemic individual. In some instances, increased L-cell secretion of enteroendocrine peptides is associated with increased insulin sensitivity.

The invention regards methods for treating obesity or diabetes comprising contacting the distal ileum of an individual in need thereof with an IBAT inhibitor of the invention.

In some embodiments of the methods, contacting the distal ileum of an individual in need thereof with an IBAT inhibitor reduces food intake, induces satiety, reduces blood and/or plasma glucose levels, treats a metabolic disorder, reduces the weight, stimulates L-cells in the distal gastrointestinal tract, increases the concentration of bile acids and salts thereof in the vicinity of L-cells in the distal gastrointestinal tract and/or enhances enteroendocrine peptide secretion of the individual.

The IBAT inhibitor may be not systemically absorbed. In other embodiments, the IBAT inhibitor is systemically absorbed.

In some embodiments, the methods described above further comprise administration of a second agent selected from a DPP-IV inhibitor, a biguanide, an incretin mimetic, a thiazolidinedione, GLP-1 or an analogue thereof, and a TGR5 agonist. In some embodiments, the second agent is a DPP-IV inhibitor.

Individual for treatment may be an obese or overweight individual, a diabetic individual or a non-diabetic individual.

The compounds and compositions of the invention may be used for the treatment of obesity and/or diabetes whereby the administration of a therapeutically effective amount of a combination of an IBAT inhibitor and a DPP-IV inhibitor is administrated to an individual in need thereof. For the treatment of obesity and/or diabetes a therapeutically effective amount of a combination of an IBAT inhibitor and a TGR5 agonist may be administrated to an individual in need thereof. In some embodiments, provided herein are methods for the treatment of obesity and/or diabetes comprising administration of a therapeutically effective amount of a combination of an IBAT inhibitor and at least one other active substance e.g. a thiazolidinedione to an individual in need thereof. Methods for the treatment of obesity and/or diabetes comprising administration of a therapeutically effective amount of a combination of an IBAT inhibitor and an incretin mimic to an individual in need thereof are also envisaged.

Obesity and/or diabetes may be treated according to the invention by administration of a therapeutically effective amount of a combination of an IBAT inhibitor and GLP-1 or an analogue thereof to an individual in need thereof. Obesity and/or diabetes may for example be treated by the administration of a therapeutically effective amount of a combination of an IBAT inhibitor and a biguanide to an individual in need thereof.

The invention further regards methods for reducing food intake in an individual in need thereof comprising administration of an IBAT inhibitor to an individual in need thereof wherein the IBAT inhibitor is delivered or released non-systemically in the distal ileum of the individual.

Reduction of food and caloric or induction of satiety in an individual in need thereof may be performed with the methods of the invention. Thus metabolic disorders may be treated and the weight be reduced of an individual in need thereof. In some embodiments, the methods described herein stimulate L-cells in the distal gastrointestinal tract of an individual in need thereof. In some embodiments, the methods increase the concentration of bile acid and salts thereof in the vicinity of L-cells in the distal gastrointestinal tract of an individual.

Circulating blood or plasma glucose levels in an individual in need thereof may be reduced by administrating of an IBAT inhibitor to an individual in need thereof wherein the IBAT inhibitor is delivered or released non-systemically in the distal ileum of the individual.

Also, insulin secretion may be increased in an individual in need thereof comprising administration of an IBAT inhibitor to an individual in need thereof wherein the IBAT inhibitor is delivered or released non-systemically in the distal ileum of the individual.

In some embodiments, the methods described herein enhance enteroendocrine peptide secretion in an individual in need thereof. In some of such embodiments, the enteroendocrine peptide is GLP-1, GLP-2, PYY, oxyntomodulin, or a combination thereof.

The distal ileum of an individual in need thereof may be brought into contact with an IBAT inhibitor and the level of GLP-1 in the blood and/or plasma of the individual increased by from about 2 times to about 7 times the level of GLP-1 in the blood and/or plasma of the individual prior to contacting the distal ileum of the individual with the IBAT inhibitor.

In some embodiments, contacting the distal ileum of an individual in need thereof with an

IBAT inhibitor reduces the level of glucose in the blood and/or plasma of the individual by at least 20%, at least 30% or at least 40% compared to the level of glucose in the blood and/or plasma of the individual prior to contacting the distal ileum of the individual with the IBAT inhibitor. Such a reduction may be kept for at least 12 or at least 24 hours compared to blood and/or plasma glucose levels in the individual prior to contacting the distal ileum of the individual with the IBAT inhibitor.

The IBAT inhibitor may be administered orally e.g. as an ileal release formulation that delivers the IBAT inhibitor to the distal ileum, colon and/or rectum of an individual. In some embodiments, the IBAT inhibitor is administered as an enterically coated formulation.

In some embodiments of the methods described above, the IBAT inhibitor is a compound of Formula I or II as described herein. In some embodiments of the methods described above, the IBAT inhibitor is a compound of Formula II as described herein.

The compounds and compositions of the invention may be administered less than about 30 e.g. less than about 60 minutes before ingestion of food. They may also be given after ingestion of food.

The invention relates to methods for prevention and/or treatment of inflammatory bowel disease, impaired bowel integrity, short bowel syndrome, gastritis, peptic ulcer, or irritable bowel disease, congestive heart failure, ventricular dysfunction, toxic hypervolemia and/or polycystic ovary syndrome, comprising contacting the distal ileum of an individual in need thereof with an IBAT inhibitor . In some embodiments, the methods further comprise administration of a DPP-IV inhibitor, a TGR5 agonist, a biguanide, an incretin mimetic, or GLP-1 or an analogue thereof. Provided herein are methods for prevention and/or treatment of radiation enteritis comprising contacting the distal ileum of an individual in need thereof with an IBAT inhibitor. In some embodiments, the methods further comprise administration of a DPP-IV inhibitor, a TGR5 agonist, a biguanide, an incretin mimetic, or GLP-1 or an analogue thereof.

The compounds and compositions may be used for reducing caloric intake in an individual in need thereof comprising an IBAT inhibitor, and a pharmaceutically acceptable carrier, wherein the IBAT inhibitor is delivered or released non-systemically in the distal ileum of the individual. Thus, compositions for reducing circulating blood and/or plasma glucose levels in an individual in need thereof may comprise an IBAT inhibitor, and a pharmaceutically acceptable carrier, wherein the IBAT inhibitor is delivered non-systemically in the distal ileum of the individual. Provided herein are compositions for increasing insulin secretion, which comprise an IBAT inhibitor, and a pharmaceutically acceptable carrier, wherein the IBAT inhibitor is delivered or released non-systemically in the distal ileum of the individual. In any of the aforementioned embodiments, the compositions further comprise a DPP-IV inhibitor, a TGR5 agonist, a biguanide, an incretin mimetic, or GLP-1 or an analogue thereof.

In some embodiments the IBAT inhibitors and the compositions comprising them are used for reducing food intake (caloric intake) or for reducing circulating blood or plasma glucose levels wherein the IBAT inhibitor is not absorbed systemically following oral administration. In some of such embodiments, the IBAT inhibitor is prevented from being absorbed in the stomach by its presence in a formulation that releases it in the ileum. In some of such embodiments, the IBAT inhibitor is administered in combination with a second therapeutic agent selected from a DPP-IV inhibitor, a biguanide, a thiazolidinedione, an increin mimetic, GLP-1 or an analogue thereof, or a TGR5 agonist.

Medicinal and Pharmaceutical use of the Invention

According to another feature of the invention there is provided an oral pharmaceutical formulation comprising an IBAT inhibitor compound or a pharmaceutically acceptable salt, solvate, solvate of such a salt or a prodrug thereof and a bile acid binder, wherein the formulation is designed to deliver the bile acid binder in the colon for use in the production of an IBAT inhibitory effect in a warm-blooded animal, such as man.

According to another feature of the invention there is provided an oral pharmaceutical formulation comprising an IBAT inhibitor compound or a pharmaceutically acceptable salt, solvate, solvate of such a salt or a prodrug thereof and a bile acid binder, and at least one of the above mentioned other active compounds and a bile acid binder of the invention wherein the formulation is designed to deliver the bile acid binder in the colon for use in prophylaxis or treatment of any of the herein mentioned medical indications in a warm-blooded animal, such as man.

According to another feature of the invention there is provided an oral pharmaceutical formulation comprising an IBAT inhibitor compound or a pharmaceutically acceptable salt, solvate, solvate of such a salt or a prodrug thereof and a bile acid binder, wherein the formulation is designed to deliver the bile acid binder in the colon for use in prophylaxis or treatment of any of the herein mentioned medical indications in a warm-blooded animal, such as man.

According to another feature of the invention there is provided an oral pharmaceutical formulation comprising an IBAT inhibitor compound or a pharmaceutically acceptable salt, solvate, solvate of such a salt or a prodrug thereof and a bile acid binder, and at least one of the above mentioned other active compounds and a bile acid binder of the invention wherein the formulation is designed to deliver the bile acid binder in the colon for use in prophylaxis or treatment of any of the herein mentioned medical indications in a warm-blooded animal, such as man.

According to another feature of the invention there is provided an oral pharmaceutical formulation comprising an IBAT inhibitor compound or a pharmaceutically acceptable salt, solvate, solvate of such a salt or a prodrug thereof and a bile acid binder, wherein the formulation is designed to deliver the bile acid binder in the colon for use in the preparation of a pharmaceutical for use in prophylaxis or treatment of any of the herein mentioned medical indications in a warm-blooded animal, such as man.

According to another feature of the invention there is provided an oral pharmaceutical formulation comprising an IBAT inhibitor compound or a pharmaceutically acceptable salt, solvate, solvate of such a salt or a prodrug thereof and a bile acid binder, and at least one of the above mentioned other active compounds and a bile acid binder of the invention wherein the formulation is designed to deliver the bile acid binder in the colon for use in the preparation of a pharmaceutical for use in prophylaxis or treatment of any of the herein mentioned medical indications in a warm-blooded animal, such as man.

In an additional feature of the invention, there is provided a method of treating any of the herein mentioned medical conditions in a warm-blooded animal, such as man, in need of such treatment which comprises administering to said animal an effective amount of an IBAT inhibitor compound or a pharmaceutically acceptable salt, solvate, solvate of such a salt or a prodrug thereof in simultaneous, sequential or separate administration with an effective amount of a bile acid binder.

In an additional feature of the invention, there is provided a method of treating any of the herein mentioned medical conditions in a warm-blooded animal, such as man, in need of such treatment which comprises administering to said animal an effective amount of an IBAT inhibitor compound or a pharmaceutically acceptable salt, solvate, solvate of such a salt or a prodrug thereof and at least one of the above mentioned other active compounds in simultaneous, sequential or separate administration with an effective amount of a bile acid binder.

Dosage Forms

The pharmaceutical compositions may be formulated as a dosage form. A dosage form may comprisea compound of formula I and II, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms optionally include an additional therapeutic compound described herein and one or more pharmaceutically acceptable additives such as a compatible carrier, binder, filling agent, suspending agent, flavouring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I-II. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

Pharmaceutical compositions may be formulated as known in the art using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions and carriers may be found, for example, in Remington: The Science and Practice of Pharmacy, Nineteenth Ed (Easton, Pa.: Mack Publishing Company, 1995); Hoover, John E., Remington's

Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A mixture of a compound of Formula I and II may optionally also comprise other active compounds and additional formulating substances, such as carriers, stabilizers, diluents, dispersing agents, suspending agents, thickening agents, and/or excipients may be used in a composition. Therapeutically effective amounts of compounds described herein may be administered in a pharmaceutical composition to an individual having a disease, disorder, or condition to be treated.The individual may be a human. The compounds may be either utilized singly or in combination with one or more additional therapeutic agents.

The pharmaceutical formulations are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

An IBAT inhibitor of Formula I and II is used in the preparation of medicaments for the prophylactic and/or therapeutic treatment of obesity and/or diabetes. A method for treating any of the diseases or conditions described herein in an individual in need of such treatment, involves administration of pharmaceutical compositions containing at least one IBAT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

A dosage form allowing for controlled release of an active agent in the distal jejunum, proximal ileum, distal ileum and/or the colon is also within the scope of the invention. In some embodiments, a dosage form comprises a polymer that is pH sensitive e.g., a MMX™ matrix from Cosmo Pharmaceuticals and allows for controlled release of an active agent in the ileum and/or the colon. Examples of such pH sensitive polymers suitable for controlled release include and are not limited to polyacrylic polymers (e.g., anionic polymers of methacrylic acid and/or methacrylic acid esters, e.g., Carbopol™polymers) that comprise acidic groups (e.g. —COOH, —SO₃H) and swell in basic pH of the intestine (e.g., pH of about 7 to about 8). In some embodiments, a dosage form suitable for controlled release in the distal ileum comprises microparticulate active agent (e.g. micronized active agent). In some embodiments, a non-enzymatically degrading poly(dl-lactide-co-glycolide) (PLGA) core is suitable for delivery of an IBAT to the distal ileum. In some embodiments, a dosage form comprising an IBAT is coated with an enteric polymer (e.g., Eudragit™S-100, cellulose acetate phthalate, polyvinylacetate phthalate, hydroxypropylmethylcellulose phthalate, anionic polymers of methacrylic acid, methacrylic acid esters or the like) for site specific delivery to the ileum and/or the colon. In some embodiments, bacterially activated systems are suitable for targeted delivery to the ileum. Examples of micro-flora activated systems include dosage forms comprising pectin, galactomannan, and/or Azo hydrogels and/or glycoside conjugates (e.g., conjugates of D-galactoside, beta-D-xylopyranoside or the like) of the active agent. Examples of gastrointestinal micro-flora enzymes include bacterial glycosidases such as, for example, D-galactosidase, beta-D-glucosidase, alpha-L-arabinofuranosidase, beta-D-xylopyranosidase or the like.

Coated units may be filled into hard gelatine capsules or mixed with tablet excipients, such as fillers, binders, disintegrants, lubricants and other pharmaceutically acceptable additives, and be compressed into tablets. The compressed tablet is optionally covered with film-forming agents to obtain a smooth surface of the tablet and further enhance the mechanical stability of the tablet during packaging and transport. Such a tablet coat, which may be applied on a multiple unit tablet or a conventional tablet, may further comprise additives like anti-tacking agents, colorants and pigments or other additives to improve the tablet appearance.

Suitable drugs for the new formulations are IBAT inhibitor compounds such as described in the above-discussed documents, hereby incorporated by references.

The IBAT inhibitor compound could alternatively be a low permeability drug as defined in the Biopharmaceutical Classification System proposed by FDA.

A combination therapy according to the invention should preferably comprise simultaneously, separately or sequentially administration of an IBAT inhibitor compound and a bile acid binder. The IBAT inhibitor could preferably be formulated for ileum delivery and the bile acid binder could preferably be formulation for colon release.

Dosage

A suitable unit dose will vary with respect to the patient's body weight, condition and disease severity. The dose will also depend on if it is to be used for prophylaxis or in the treatment of severe conditions, as well as the route of administration. The daily dose can be administered as a single dose or divided into one, two, three or more unit doses. An orally administered daily dose of an IBAT inhibitor is preferably within 0.1 -1,000 mg, more preferable 1 -100 mg.

A pharmaceutical formulation according to the present invention with a targeted delivery in the gastro intestinal tract provides a reduced systemic exposure, as can be measured by the area under the drug plasma concentration versus time curve (AUC) or 7a-hydroxy-4-cholesten-3-one (C4), while maintaining or even increasing the therapeutic effect, as e.g. measured by serum cholesterol reduction.

A combination therapy comprising an IBAT inhibitor and a bile acid binder comprises preferably a low daily dose of the bile acid binder, such as less than 5 g of a resin, and more preferably less than 2 g. A dosage form with colon release of the bile acid binder could be constructed by any of the above described principles for delayed release formulations.

A combination therapy comprising an IBAT inhibitor and a bile acid binder may comprise a low daily dose of the bile acid binder, such as less than 5 g of a resin, and more preferably less than 4, 3, 2 or less than 1 g. Suitable ranges may be 0,1-5 g, 0.5-4 g, 1-3 g, 2-4 g, 2-3 g per day. A dosage form with colon release of the bile acid binder could be constructed by any of the above described principles for delayed release formulations.

A tablet may consist of an inner core of 1-1000 mg, e.g. 200-800 mg, 50-400 mg, 10-200 mg or 20-80 mg acid binder in a colonic delivery formulation and an outer lamina with 1-100 mg, 5-50 mg e.g. 1-20 mg of an IBAT inhibitor.

The daily dose of IBAT inhibitor and /or bile acid binder can be administered as a single dose or divided into one, two, three or more unit doses.

Dosing three times a day with 400 mg of colesevelam in a colonic release formulation will give an adequate binding of bile acids in the colon as the total luminal volume is expected to be about 100 ml, which is in accordance to an accepted pharmacokinetic calculation volume of 250 to 300 ml for the small gut. The daily recommended total dose of colesevelam to block bile acid absorption in total gut of humans is 3750 mg/day.

The invention also regards a method for treatment and/or prophylaxis of obesity or diabetes, in a warm-blooded animal, such as man, in need of such treatment and/or prophylaxis comprising administering an effective amount of a compound or a composition according to the invention to the individual.

A method for treating any of the diseases or conditions described herein in an individual in need of such treatment, may involve administration of pharmaceutical compositions containing at least one IBAT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

Further, the invention relates to a kit comprising compound or a composition according to the invention and optionally also an instruction for use.

The following contemplated Examples are intended to illustrate, but in no way limit the scope of the invention. All references cited herein are hereby incorporated by reference in their entirety.

The expression “comprising” as used herein should be understood to include, but not be limited to, the stated items.

### Example 1

1,1-Dioxo-3,3-dibutyl-5-phenyl-7-methylthio-8-(N-{(R)-α-[N-(carboxymethyl)carbamoyl]benzyl} carbamovlmethoxv)-2,3,4,5-tetrahvdro-1. 2. 5-benzothiadiazepine, Mw. 696,89.

This compound is prepared as described in Example 2 of WO3022286.

### Example 2

1,1-Dioxo-3, 3-dibutyl-5-phenyl-7-methylthio-8-(N-{(R)-α-[N′-((S)-1-carboxyethyl) carbamoyl] benzyl} carbamoylmethoxy)-2, 3,4, 5-tetrahydro-1, 5-benzothiazepine, Mw. 709,92.

This compound is prepared as described in Example 2 of WO03106482.

### Example 3

1,1-Dioxo-3,3-dibutyl-5-phenyl-7-methylthio-8-(N-{(R)-α-[N-((S)-1-carboxypropyl) carbamoyl]benzyl}carbamoylmethoxy)-2,3,4,5-tetrahydro-1,2,5-benzothiadiazepine, Mw. 724,94.

This compound is prepared as described in Example 6 of WO3022286.

### Example 4

1,1-Dioxo-3,3-dibutyl-5-phenyl-7-methylthio-8-(N-{(R)-α-[N-((R)-1-carboxy-2-methylthioethyl)carbamoyl]benzyl}carbamoylmethoxy)-2,3,4,5-tetrahydro-1,2,5-benzothiadiazepine, Mw. 757,01.

This compound is prepared as described in Example 7 of WO3022286.

### Example 5

1,1-Dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine, Mw. 740,94.

This compound is prepared as described in Example 29 of WO3022286.

### Example 6

1,1-Dioxo-3,3-dibutyl-5-phenyl-7-methylthio-8-(N-{(R)-α—N-((R)-1-carboxy-2-methylthio-ethyl)carbamoyl]-4-hydroxybenzyl}carbamoylmethoxy)-2,3,4,5-tetrahydro-1,2,5-benzothiadiazepine, Mw. 773,00.

This compound is prepared as described in Example 30 of WO3022286.

### Example 7

1,1-Dioxo-3,3-dibutyl-5-phenyl-7-methylthio-8-(N-{(R)-α-[N-((S)-1-carboxy-2-methylpropyl)carbamoyl]benzyl}carbamoylmethoxy)-2,3,4,5-tetrahydro-1,2,5-benzothiadiazepine, Mw. 738,97.

This compound is prepared as described in Example 15 of WO3022286.

### Example 8

1,1-Dioxo-3,3-dibutyl-5-phenyl-7-methylthio-8-(N-{(R)-α-[N-((S)-1-carboxy-2-(R)-hydroxypropyl)carbamoyl]-4-hydroxybenzyl}carbamoylmethoxy)-2,3,4,5-tetrahydro-1,2,5-benzothiadiazepine, Mw. 756,94.

This compound is prepared as described in Example 26 of WO3022286.

### Example 9

1,1-Dioxo-3,3-dibutyl-5-phenyl-7-methylthio-8-(N-{(R)-α-[N-((S)-1-carboxybutyl) carbamoyl]-4-hydroxybenzyl}carbamoylmethoxy)-2,3,4,5-tetrahydro-1,2,5-benzothiadiazepine, Mw. 754,97.

This compound is prepared as described in Example 28 of WO3022286.

### Example 10

1,1-Dioxo-3,3-dibutyl-5-phenyl-7-methylthio-8-(N-{(R)-α-[N-((S)-1-carboxyethyl) carbamoyl]benzyl}carbamoylmethoxy)-2,3,4,5-tetrahydro-1,2,5-benzothiadiazepine, Mw. 710,91.

This compound is prepared as described in Example 5 of WO3022286.

### Example 11

1,1-Dioxo-3, 3-dibutyl-5-phenyl-7-methylthio-8-(N-{(R)-α-[N′-((S)-1-carboxypropyl) carbamoyl]-4-hydroxybenzyl} carbamoylmethoxy) -2, 3,4, 5-tetrahydro-1, 5-benzothiazepine, Mw. 739,95.

This compound is prepared as described in Example 1 of WO3022286.

### Example 12

1,1-Dioxo-3,3-dibutyl-5-phenyl-7-methylthio-8-(N-{(R)-α—N-((S)-1-carboxyethyl)carbamoyl]-4-hydroxybenzyl}carbamoylmethoxy)-2,3,4,5-tetrahydro-1,2,5-benzothiadiazepine, Mw. 726,91.

This compound is prepared as described in Example 11 of WO3022286.

### Example 13

1,1-Dioxo-3,3-dibutyl-5-phenyl-7-methylthio-8-(N-{(R)-α-[N-((S)-1-carboxy-2-methylpropyl)carbamoyl]-4-hydroxybenzyl}carbamoylmethoxy)-2,3,4,5-tetrahydro-1,2,5-benzothiadiazepine, Mw. 754,97.

This compound is prepared as described in Example 27 of WO3022286.

### Example 14

1,1-Dioxo-3, 3-dibutyl-5-phenyl-7-methylthio-8-(N-{ (R)-1′-phenyl-1′-[N′-(carboxymethyl) carbamoyl] methyl} carbamoylmethoxy)-2, 3,4, 5-tetrahydro-1, 5-benzothiazepine, Mw. 695,90.

This compound is prepared as described in Example 43 of WO0250051.

### Example 15

Pharmaceutical Effect Mean Inhibitory Effect (%)

ISBT Hu HEK Uptake SPA 13203 IBAT HUM Ileal Bile Acid Transporter Human HEK Glycocholic acid Uptake Radiometric—SPA Inhibitor IC50 Mean IC50 (nM) was determined for the compounds of examples 1-14

Test System

Animals

Species Mouse; Strain ApoE knock out; Substrain C57BL/6; Sex Female; Total No. of animals 70; Body weight range 20 g to 22 g; Supplier Mollegaard's Breeding (Skensved, Denmark); Identification method ID cards (bar code).

Acclimatisation At least one week at the Section of Laboratory; Animal Resource at AstraZeneca; Housing conditions Kept five by five in cages (Makrolon III, 7 dm2) in a room with regulated temperature (220 C.), relative humidity (40% to 60%) and a 12/12 hours light/dark cycle. Diet Free access to R3 pellets (Lactamin, Vadstena, Sweden) during the housing and experimental period. Water Free access to tap water during the housing and experimental period.

Study Design

Dose(s) 0.156 pmol/kg

0.625 pmol/kg

2.5 pmol/kg

Volume(s) of administration 0.1 mL per mousen

Route(s) and frequency of administration

Oral administration and single dose

Number/group 3 mice per group

Number of groups 4 groups

Experimental Procedures

The animals were orally administered vehicle or Example 14 (0.156 , 0.625 or 2.5 pmol/kg) at 13:00 o'clock on the experimental day. Thirty minutes later, a trace amount of 75SeHCAT (0.1 mCi per 0.1 mL per mouse) was orally given to each mouse. Twenty-four hours after 75SeHCAT administration, the animals were killed by CO2 inhalation. At sacrifice, the gall bladder and the whole intestine were removed, and the faeces during the 24-hour period after 75SeHCAT administration was collected for each mouse. The gamma radioactivities of 75SeHCAT in the faeces and in the gall bladder-intestine were separately counted by 1282 CompuGamma CS Gamma counter (Wallac oy, Turku, Finland). The stability as well as the quantity of the 75SeHCAT administered to each mouse, were controlled with an additional 75SeHCAT aliquot following the same experimental process as other tested samples in the study.

Data Analysis

The sum of the gamma counts from both the faeces and the gall bladder-intestine was considered as the total recovered 75SeHCAT, which was averaged around 85% of the total 75SeHCAT administered to each mouse. Of the recovered radioactivity of 75SeHCAT, the percentage of the 75SeHCAT detected in the faeces was considered as the faecal excretion while that in the gall bladder-intestine as body retention. Inhibitory effect of Example 14 IBAT inhibitor on 75SeHCAT intestinal absorption was calculated following the 75SeHCAT body retention and the faecal excretion, and the ED50 of the compound was estimated following the dose-effect curve.

Results

The mean IBAT inhibitory effect (%) at a dose (pmol/kg): 0.156 was determined for the compounds of examples 1-14 and is reported in Table 1.

TABLE 1

| | | % inhibition | Mean IC50 |
|---|---|---|---|
| Example | Structure | 0.156 μmol/kg | nM |
| 1. | 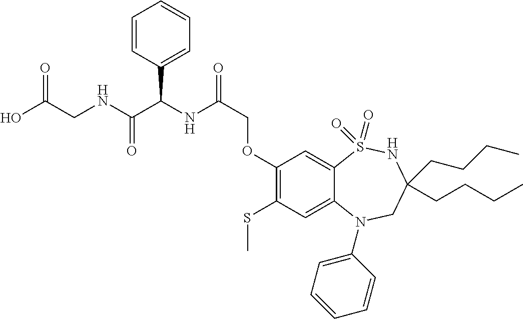 | 43 | 0.45 |
| 2. | 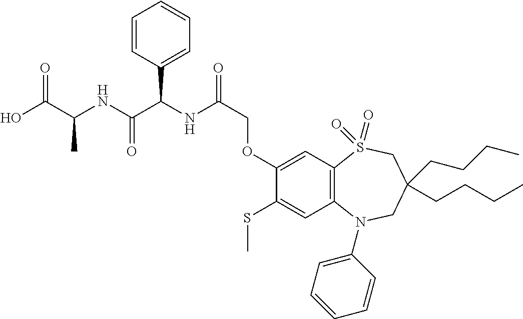 | 55 | 0.39 |
| 3. | 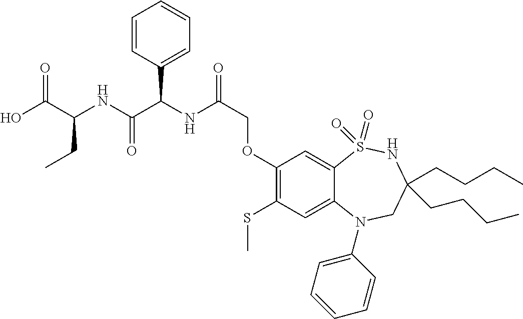 | 63 | 0.18 |
| 4. | 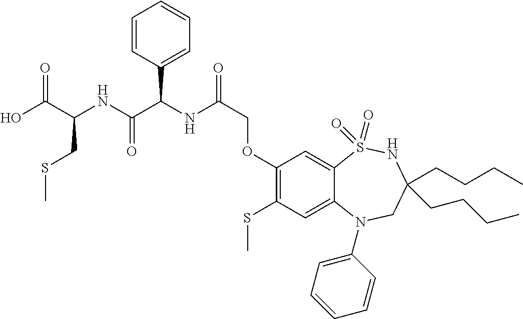 | 63 | 0.35 |
| 5. | 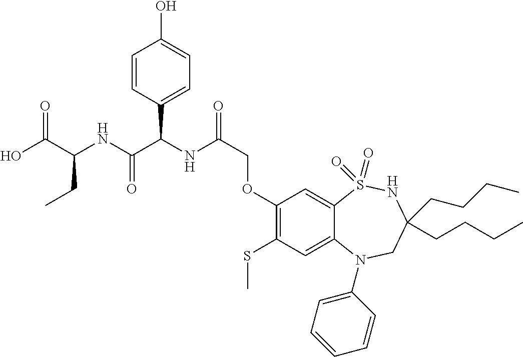 | 74 | 0.16 |
| 6. | 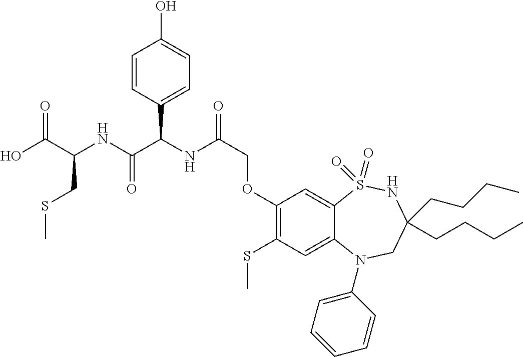 | 59 | |
| 7. | 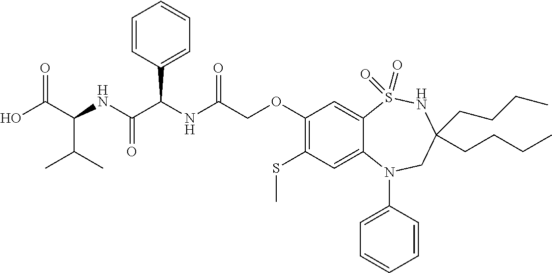 | 66 | 0.36 |
| 8. | 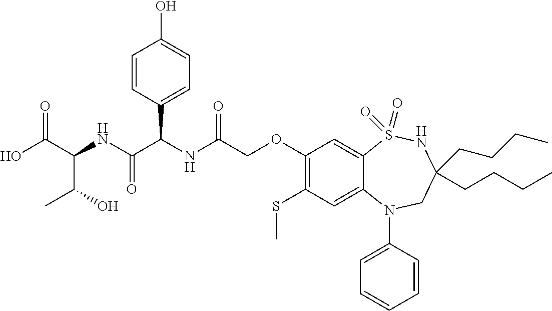 | 46 | 0.11 |
| 9. | 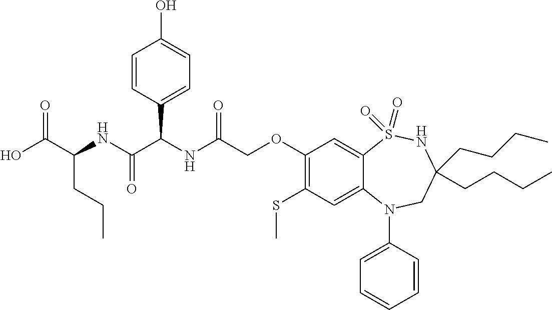 | 67 | |
| 10. | 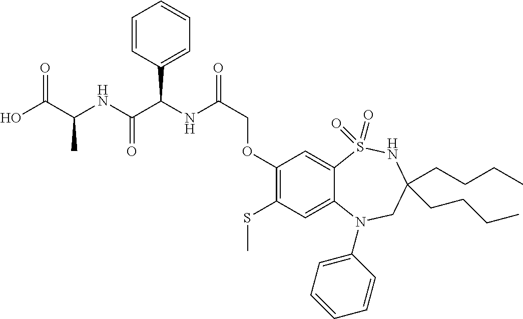 | 68 | 0.2 |
| 11. | 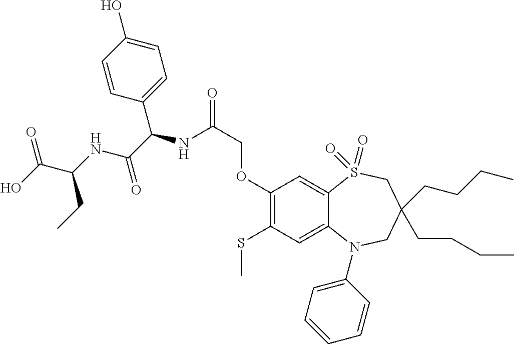 | 63 | 0.15 |
| 12. | 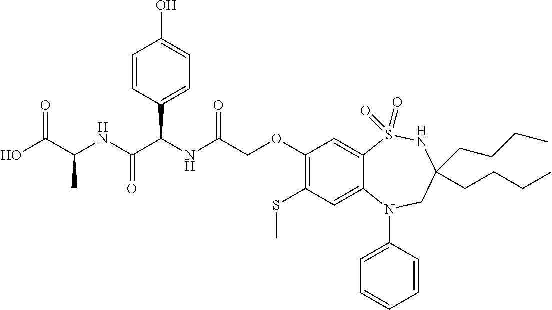 | 63 | 0.3 |
| 13. | 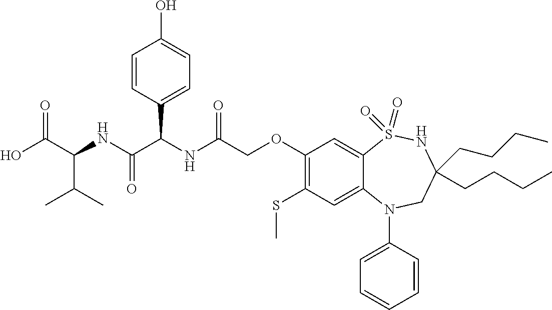 | 68 | |
| 14. | 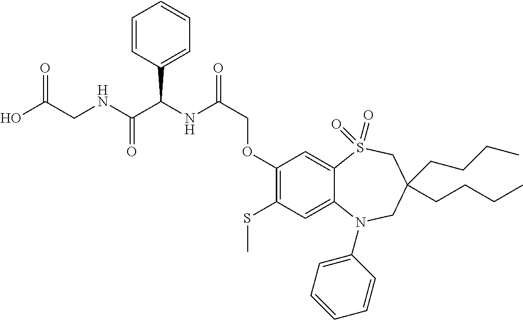 | 28 | 1.2 |

### Example 16

Interruption of bile acid circulation by inhibition of the bile acid transporter Slc 10a2 improves triglyceride metabolism and normalizes plasma glucose levels.

Expermintal Procedures

Animals

Slc10a2+/− and Slc10a2−/− mice were generated at AstraZeneca R&D, Molndal, as described below and as outlined by Dawson et al (2).

Targeting the Slc10a2 Locus

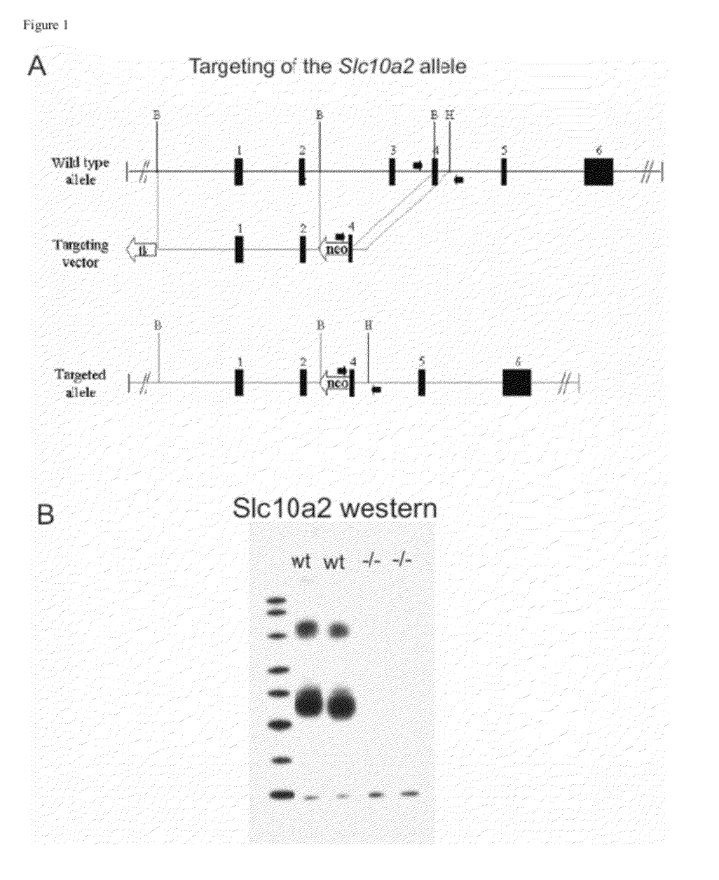

The targeting vector used to modify the mouse Slc10a2 locus was a kind gift from P. Dawson and has been previously described (2). In brief, it consisted of a˜14kb 5′homology arm, an inverted Neo (neomycin phosphotransferase) cassette driven off the PGK (phosphoglycerate kinase) promoter and a 1.6 kb 3′ homology arm. The targeting vector was designed so that correct targeting would result in most of intron 2, exon 3, intron 3 and the very 5′ end of exon 4 being deleted and replaced by the Neo cassette in order to inactivate the Slc10a2 gene (see FIG. 1A). B, BamHI; H, HeeIII. After linearization, the targeting construct was electroporated into R1 ES cells (derived from 129/SvJ) and neomycin-resistant clones were selected in G-418-containing (300 pg/ml) media. Of 400 G418-resistant clones screened, 2 targeted clones were identified using a PCR screening over the short arm and then confirmed by Southern analysis. The primers used for detecting the targeted allele were a forward primer located in the inverted Neo cassette and a reverse primer located downstream of the short arm (5′-cgtactggggcatagaatctttgc-3′). The same reverse primer was combined with a forward primer in intron 3 (5′-ctcttcctatgaagctaaaggggc-3′) for detection of the wild-type allele. One positive clone was expanded and injected into C57B1/6 blastocysts to generate chimeric mice. Chimeric males were backcrossed to

C57B1/6 females and genotyping of the offspring was performed from tail biopsies by both

PCR and Southern to confirm germline transmission. To verify that the targeted SLC10A2 allele resulted in a null mutation, total RNA was prepared from the kidneys and intestines of 8-10 week old homozygous, heterozygous, and wild-type littermates using TRIzol Reagent according to the manufacturer's instructions (Invitrogen, Paisley, UK). cDNA was synthezised using Super-script II Rnase H-Reverse Transcriptase and random hexamer primers (Invitrogen, Paisley, UK). TaqMan real-time PCR was performed using the ABI PRISM 7700 Sequence Detector System (Applied Biosystems, Warrington, UK). All samples were run in triplicate and data were normalized using the mouse acidic ribosomal phosphoprotein PO (M36B4) as an internal control. The TaqMan primers and probe for SLC10A2 were: 5′-accacttgctccacactgctt-3′(forward), 5′-acccacatcttggtgtagacga-3′(reverse) and 5′-ccttggaatgatgcctctttgcctc-3′ (probe).

A diet enriched in sucrose (D12329, Research Diets, NJ) together with drinking water supplemented with 10% fructose was used for the high carbohydrate experiment. Animals had free access to the diet for two weeks. Control animals received standard mouse chow and tap water. Male ob/ob animals were from Taconic, DK. Ob/ob animals were gavaged with a specific Slc10a2 inhibitor Example 14 or a control vehicle for 11 days. Animals had free access to food and water. All animal care and experiments were conducted in accordance with accepted standards of humane animal care and approved by the Ethics Committee of Goteborg University.

Plasma Analysis

Blood was centrifuged, and plasma was analyzed for total cholesterol and TGs using the IL TestTM cholesterol 181618-10 and TG 181610-60 kits on the Monarch 2000 system (IL Scandinavia, Gothenburg, Sweden). Lipoprotein cholesterol profiles were obtained by separation of 10 pl of plasma using a micro fast protein liquid chromatography (FPLC) system for the generation of lipoprotein profiles (15). Plasma insulin levels were analyzed using a rodent insulin RIA kit (Linco, St. Charles, Mich.). Total plasma glucose was analyzed using the IL Test (IL Scandinavia, Gothenburg, Sweden) on the Monark 2000 system. Blood glucose was determined using a Bayer Elite glucometer (Bayer diagnostics, Germany). Plasma free fatty acids were analyzed employing a commercial 3 NEFA kit (Wako Chemicals

USA Inc. Richmond, Va.). Serum levels of 7a-hydroxy-4-cholesten-3-one (C4) were used as an indirect measurement of CYP7A1 activity, and analyzed in either pooled or individual plasma samples by high pressure liquid chromatography (16).

Enzymatic Activities

HMGCoA reductase and CYP7A1 enzymatic activities were assayed in hepatic microsomes as described (17).

RNA Extraction and Quantitative Real Time PCR

Total RNA was extracted from frozen livers or distal ileum with TRIzol reagent (Invitrogen, Carlsbad, Calif.). The RNA was DNase-treated with RQ1 Dnase (Promega, Madison, Wis.). cDNA synthesis and quantitative real time PCR was performed employing HPRT as endogenous control (18). Primer and probe information are available upon request.

Liver Protein and Immunoblotting

A ligand blot employing ¹²⁵I-labeled rabbit β-VLDL was used to detect LDL receptors in liver as described (19). Hepatic SR-BI was assayed by immunoblot using a rodent specific rabbit polyclonal antibody (Novus Biologicals Inc., Littleton, Colo.), as described (20). Cyp7a1 protein levels were assayed in liver microsomal protein samples by immunoblot using a rabbit polyclonal antibody directed against the C-terminus of the CYP7A1 (18). To detect SREBP1 protein, cytoplasmic and nuclear protein preparations from liver were performed using the NE—PER reagent (Pierce), including Complete protease inhibitor (Roche), 1 mM phenyl-methylsulfonyl fluoride, 0.5 mM leupeptin, 5 μg/ml Calpain inhibitor I (Biomol, Pa.), following the manufacturer's instructions. 50 μg and 25 μg of cytoplasmic and nuclear liver protein fractions, respectively, were electrophoresed on NuPage Bis-Tris gels (Invitrogen) and transferred to nitrocellulose membranes. Membranes were blocked in 5% skimmed milk powder and incubated with a mouse monoclonal antibody raised against the N′-terminus of SREBP1 (Labvision Corporation, Calif.) at 1.5 μg/mL in 5% skimmed milk powder for two hours at room temperature. An HRP conjugated goat anti-mouse F(ab)2 antibody (Pierce) was used for detection of specific signals together with Supersignal reagent (Pierce) and a Fuji BAS 1800 analyzer (Fuji Photo Film Co.). To analyze phosphorylated liver proteins by western blot, total liver protein homogenates were prepared from frozen tissue by homogenization using a polytron followed by sonication in a buffer containing 20 mM Tris-Hcl, pH 7,4, 1% Triton X-100, 10% Glycerol, 150 mM NaCl, 2 mM EDTA, 25 mM betaglycerophosphate, 20 mM sodium floride, 1 mM sodium orthovanadate, 2 mM sodium pyrophosphate, 1 mM benzamidine, 1 mM phenyl-methylsulfonyl fluoride, 0.5 mM leupeptin, Complete proteinase inhibitor (Roche), and then centrifuged at 14 000 rpm in a microcentrifuge, and the supernatant was recovered. 50 μg protein was loaded on NuPage Bis-Tris gels (Invitrogen) and transferred to nitrocellulose membranes. Membranes were blocked in Starting Block—PBS (Pierce) and incubated in 3% BSA with phosphorylation site specific antibodies, pAkt1, pErk1/2, pMek1/2, pAmpk as indicated in figures overnight at +4 C, then stripped with Restore (Pierce) and reprobed with antibodies detecting total amounts of Akt1, Mek1/2, Erk1/2, and Ampk and finally stripped and reprobed with an antibody against beta-actin (Abcam) to verify gel loading. Antibodies against kinases were purchased from Cell Signaling Technology, Inc. Blots were developed as described above.

Determination of Liver TGs and Cholesterol

Liver cholesterol was determined as previously described and liver TGs were extracted (21) and determined employing a commercially available kit (Roche Applied Science, Indianapolis).

Statistics

Data show mean +/− SEM. The significance of differences between groups was tested by 1-way ANOVA followed by post-hoc comparisons according to Dunnett using GraphPad PrismSoftware. For the studies on ob/ob mice, the significance between groups was tested by Student's t-test.

### RESULTS

Generation of Slc10a2−/− mice

Generation of Slc10a2−/− mice was performed as described in detail in FIG. 1 experimental procedures. Southern blotting, quantitative real time PCR and immunoblotting confirmed appropriate targeting and lack of Slc10a2 expression in the null mice (FIG. 1 and not shown.) Slc10a2−/− animals were viable and fertile. No abnormalities in behaviour, gross appearance or survival were seen, consistent with the previous report by Dawson et al. (2).

Increased BA synthesis in male Slc10a2+/− and Slc10a2−/− mice

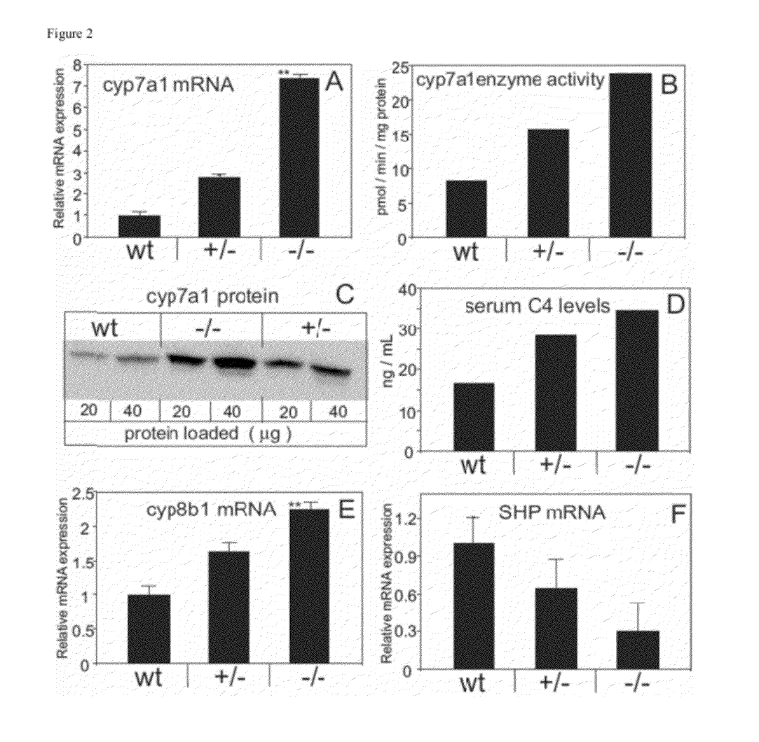

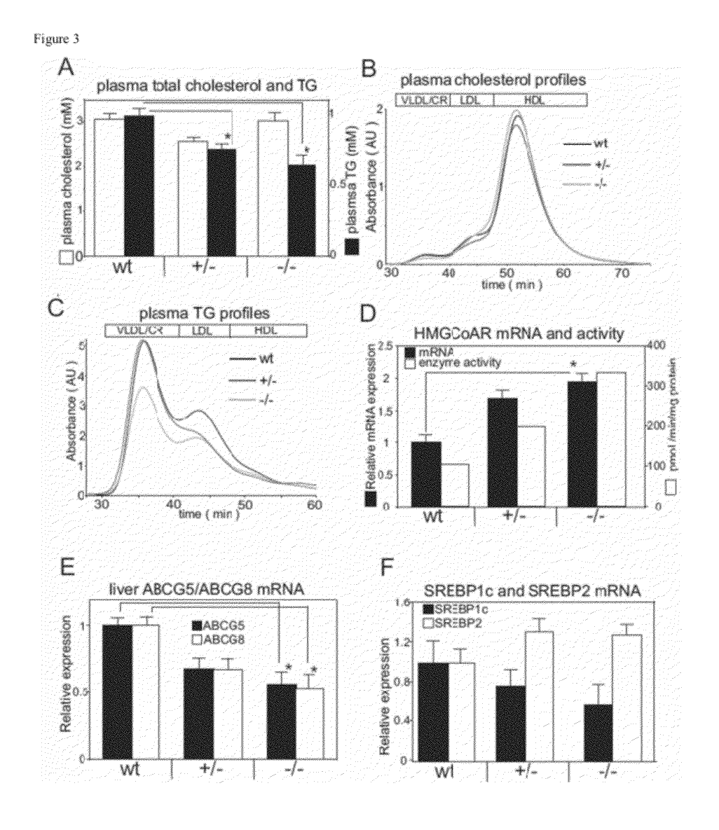

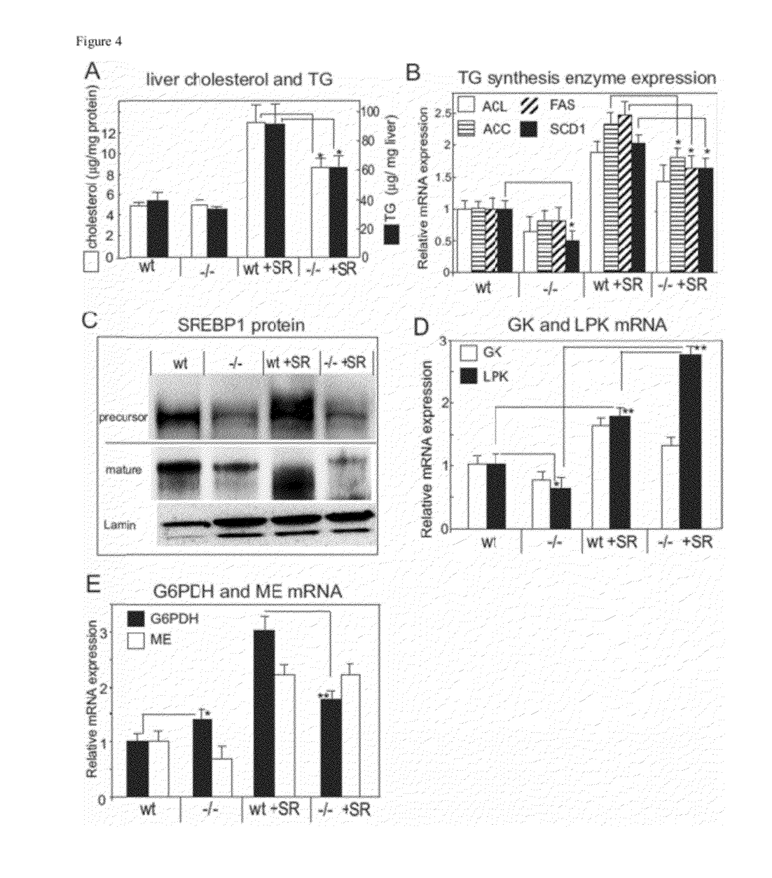

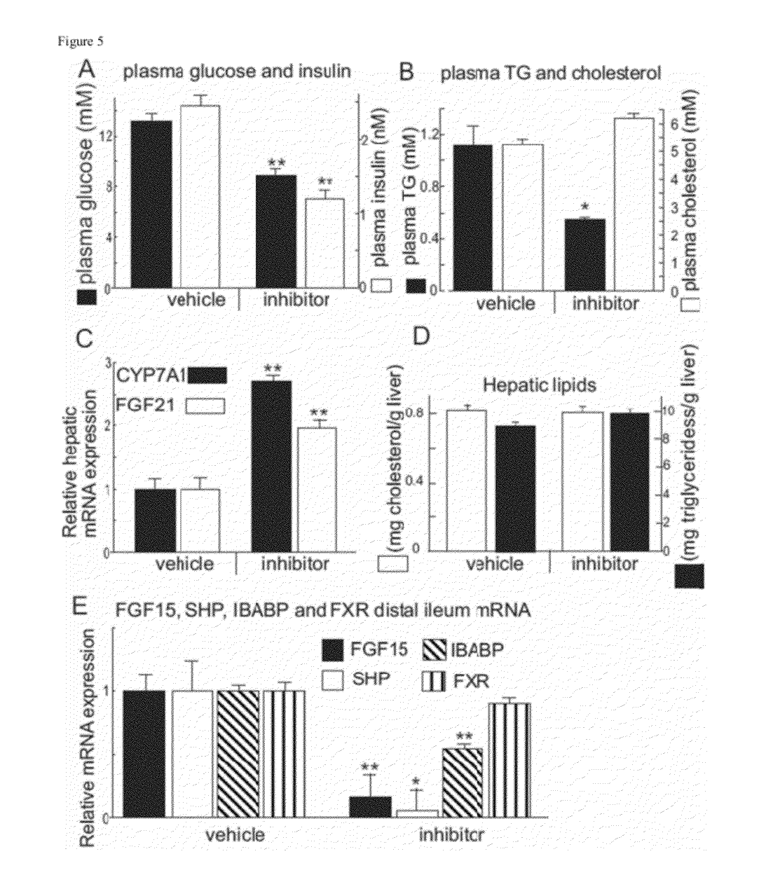

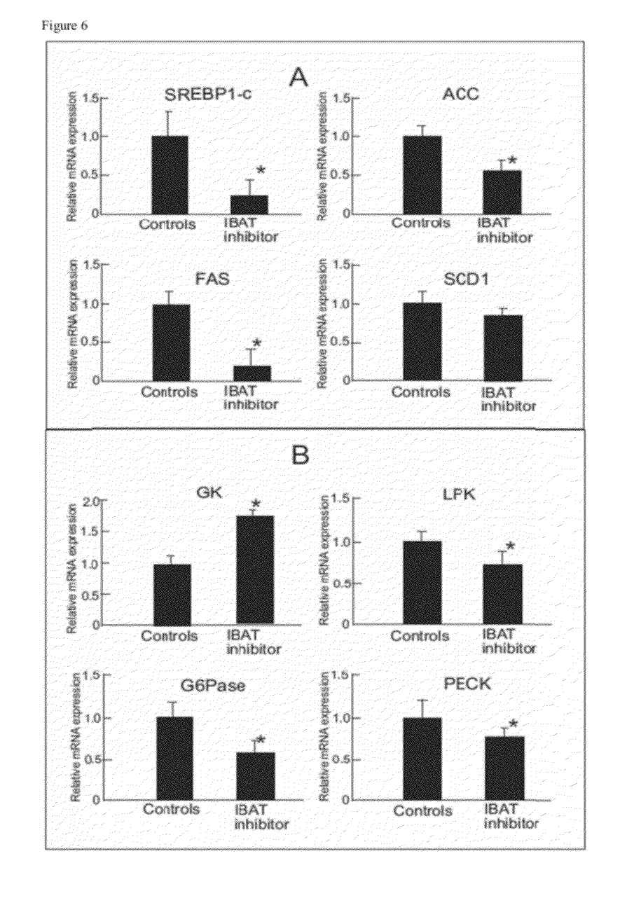

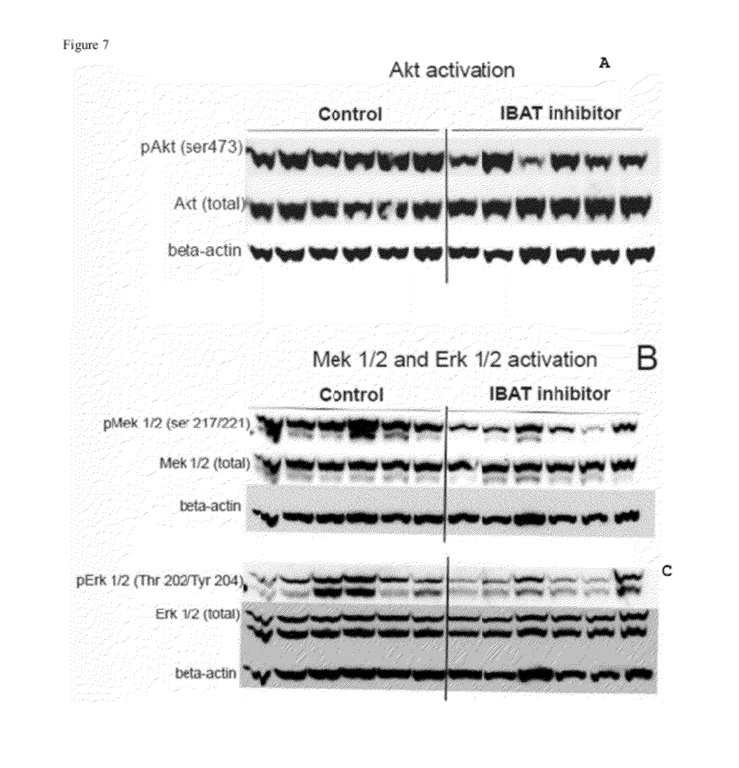

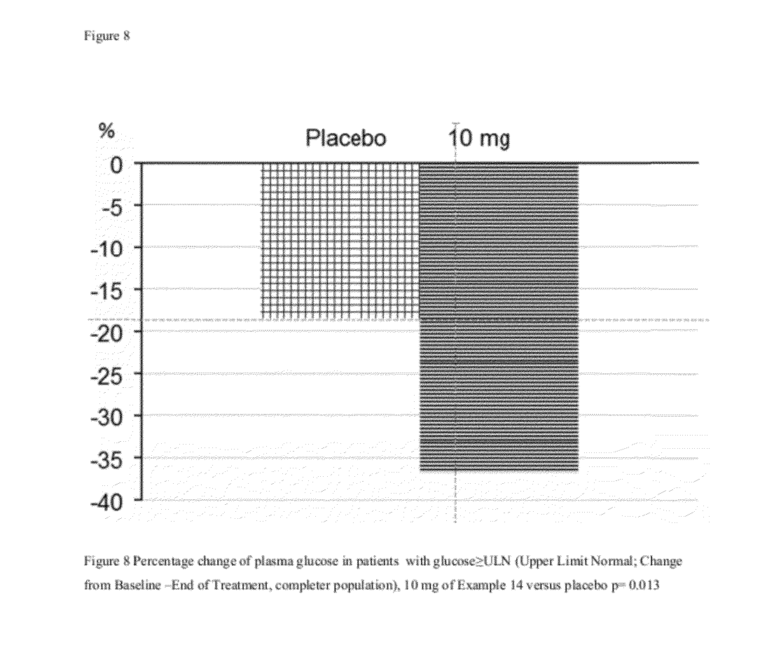

An established response following disruption of the enterohepatic circulation of BAs is an induced synthesis of BAs (2,6-8,22). Indeed, when the hepatic mRNA levels for CYP7A1, the rate-limiting enzyme in the synthesis of BAs, were assayed by quantitative real time PCR (qrtPCR), there was an ˜7-fold induction in Slc10a2−/− mice (FIG. 2A). Also, in the Slc10a2+/− mice there was a clear but less pronounced (-3-fold) increase in CYP7A1 mRNA. Consistent with the CYP7A1 mRNA increase, the enzymatic activity and the protein mass of CYP7A1 were higher in both Slc10a2+/− and −/− mice, when examined in pooled microsomes (FIGS. 2B and 2C). The CYP7A1 reaction product 7α-hydroxy-4-cholesten-3-one (C4), present in blood serum, has been demonstrated to be an accurate marker of CYP7A1 enzyme activity (16). Analysis of pooled serum from both heterozygous and homozygous animals revealed higher serum C4 levels as compared to control animals (FIG. 2D). In conjunction with the observed increase in CYP7A1, the mRNA levels for the 12 alphahydroxylase, CYP8B1, were also induced dosedependently (FIG. 2E). When the circulation of BAs is disrupted, the amount of available ligand for the hepatic nuclear BA receptor FXR decreases. In line with this, decreased hepatic mRNA levels of the FXR target gene, Small Heterodimer Partner (SHP), a suppressor of CYP7A1 gene transcription, were found in heterozygous and homozygous mice, with the most drastic change in the latter group (FIG. 2F).

Lowered plasma TGs in Slc10a2+/− and Slc10a2−/− mice

Plasma total TGs were significantly reduced by 22% in Slc10a2+/− mice and by 35% in Slc10a2−/− mice compared to controls (FIG. 3A). The plasma cholesterol and TG lipoprotein profiles were then analyzed by fast performance liquid chromatography (FPLC) (FIGS. 3B and C). The plasma cholesterol profiles did not vary notably between controls and homozygous animals. However, larger changes were found in the plasma TG profiles of these mice (FIG. 3C). In Slc10a2+/− mice, TGs were reduced within LDL, whereas in Slc10a2−/− mice TGs were reduced in both VLDL- and LDL (FIG. 3C). Plasma glucose and insulin were however not altered in Slc10a2−/− animals in this experiment (not shown).

Adaptation of Hepatic Cholesterol Metabolism to BA Deficiency

There were no changes in the hepatic expression of the LDL-receptor or the HDL-receptor SR-BI, neither at mRNA nor at protein levels (data not shown). The increased need for cholesterol as substrate for CYP7A1 in the livers of Slc10a2+/− and Slc10a2−/− mice was reflected in increased enzymatic activity of the hepatic HMGCoAreductase, with a 2-fold increase in the heterozygous and a 3.5-fold increase in homozygous animals, based upon analysis of pooled microsomal samples (FIG. 3D). The increases in hepatic HMGCoA reductase mRNA levels had a similar pattern, although smaller differences were observed between groups (FIG. 3D). The gene expression of the hepatic sterol transporters ABCG5 and ABCG8 were suppressed by up to 50% in a gene-dose dependent manner (FIG. 3E). In line with the findings for HMGCoA reductase, the hepatic levels of SREBP2 mRNA were increased in both Slc10a2+/− and Slc10a2−/− mice (FIG. 3F). Intriguingly, the regulation of the SREBP1 c gene in the liver displayed an opposite pattern, with reduced mRNA levels in heterozygous and homozygous mice compared to controls, again in a gene-dose dependent manner (FIG. 3F).

Hepatic TG production is suppressed in Slc10a2−/− mice

To further explore TG metabolism in this animal model of BA malabsorption, Slc10a2−/− and wt control animals received a sucrose-rich diet (SRdiet), to increase substrate availability, for two weeks while animals on chow served as controls. Neither plasma glucose, insulin nor food intake were significantly different between Slc10a2−/− and respective wt control group, for both diet types used (data not shown). Likewise, a densitometric x-ray analysis (DEXA) did not reveal any significant changes in body composition of Slc10a2−/− animals as compared to wt controls (not shown). The hepatic TG content tended to be lower in Slc10a2−/− mice fed regular chow (FIG. 4A). This difference was more evident on the SR diet; hepatic TGs increased by 120% in wt animals, whereas in Slc10a2−/− animals this increase was significantly blunted (FIG. 4A). Also, hepatic cholesterol was lower in Slc10a2−/− mice fed the SR diet compared to wt controls on this diet (Fig.4A). Since the lower hepatic TG levels in Slc10a2−/− mice could be due to decreased fatty acid synthesis the mRNA levels of ACL, ACC, FAS and SCD1 by qRT PCR were measured The gene expression of these enzymes was reduced in the livers of Slc10a2−/− animals (FIG. 4B); this finding was more pronounced when animals were challenged with the SR diet. The transcription factor SREBP1c is crucial for optimal activation of most genes in the fatty acid synthesis pathways (23). The protein expression of SREBP1c (mature and precursor form) was reduced in the Slc10a2−/− mice, as evaluated by Western blot on cytoplasmic and nuclear protein fractions using an antibody against the N′-terminus of SREBP1c (FIG. 4C).

Disruption of BA Circulation Alters the Expression of Genes Involved in Hepatic Glucose Handling

Hepatic TG synthesis is dependent on substrate flow in the glycolytic pathway and thus on the activity and gene expression of glucose metabolizing enzymes (24). The mRNA levels of the hepatic glycolytic enzyme glucokinase (GK) were unaltered in Slc10a2−/− animals as compared to wt controls both on chow and on the SR diet (FIG. 4D). However, the mRNA levels of liver pyruvate kinase (LPK) were reduced by 30% in Slc10a2−/− on chow (FIG. 4D). Interestingly, feeding the SR diet to Slc10a2−/− mice resulted in a 4.3-fold upregulation of LPK mRNA from basal levels, whereas in wt control animals there was a more modest 1.8-fold stimulation under the same conditions. Determination of the NADPH generating enzyme glucose-6-phosphate dehydrogenase (G6PDH) mRNA showed increased levels inSlc10a2−/− animals as compared to wt mice on regular chow (FIG. 4E).

Feeding the SR diet resulted in a three-fold increase of G6PDH mRNA in wt mice whereas in Slc10a2−/− mice there was only a modest 60% increase (FIG. 4E). It was therefore of interest to also determine if the gene expression of malic enzyme (ME), also part of a NADPH generating step converting malate to pyruvate was changed in Slc10a2−/− livers. As seen from FIG. 4E, ME mRNA tended to be decreased in Slc10a2−/− mice on regular chow and increased during the SR diet to similar extents in wt and Slc10a2−/− animals.

Inhibition of Slc10a2 in ob/ob mice reduces plasma glucose and TGs in conjunction with increased hepatic FGF21 mRNA.

Although serum glucose in Slc10a2−/− mice was not changed, these animals were hypolipidemic. It was therefore evaluated if beneficial effects could be obtained in ob/ob mice, a model where plasma glucose and TGs are chronically elevated. Ob/ob mice were treated with the specific Slc2a10 inhibitor Example 14 for 11 days. Fasting levels of glucose were reduced by 30% in drug-treated ob/ob mice compared to vehicletreated animals (FIG. 5A). Likewise, this treatment reduced fasting insulin levels by 50% in drug-treated animals. Also, the total plasma TG levels were reduced by 73% in drug-treated controls, whereas total plasma cholesterol tended to be slightly (16%) increased (FIG. 5B). It was previously found that the mRNA levels of the hormone FGF21 are increased 10-fold in ob/ob livers compared to that of wt animals (25). In addition, administration of exogenous FGF21 reduces serum TGs, insulin and glucose (26). Interestingly, the hepatic mRNA expression of FGF21 was doubled in mice treated with the Slc10a2 inhibitor (FIG. 5C). As expected, the hepatic mRNA level of CYP7A1 was increased in drug-treated mice (FIG. 5C). When the hepatic cholesterol and TG content was analyzed no difference was seen between inhibitor-treated and control animal (FIG. 5D). To verify Slc10a2 blockade, the mRNA levels of the FXR target genes FGF15, SHP and IBABP were assayed in samples from the distal ileum; they were all significantly reduced in response to treatment as could be expected from a diminished influx of BAs into the distal ileum (FIG. 5E). The gene expression for FXR was unaltered in the distal ileum following treatment with the Slc10a2 protein inhibitor (FIG. 5E).

Inhibition of Ileal Slc10a2 in ob/ob Mice Reduces SREBP1c and its Target Genes in the Liver

Since dysregulated SREBP1c has been reported to be a major determinant of the increased lipogenic response in ob/ob liver, ultimately leading to reduced hepatic insulin sensitivity, the mRNA expression of hepatic SREBP1c was next analyzed and three of its target genes. In accordance with the reduced SREBP1c levels observed in Slc10a2−/− mice both under basal and SR diet challenge, markedly reduced mRNA levels of SREBP1c were observed in response to treatment (FIG. 6A). Moreover, the mRNA levels of the SREBP1c liver target genes ACC and FAS were strongly decreased, by 50% and 80%, respectively (FIG. 6A). However, the SCD1 mRNA level was not significantly altered compared to the vehicle-treated controls (FIG. 6A).

Altered Expression of Hepatic Glucose Metabolic Genes in Slc10a2-Inhibitor Treated ob/ob Mice

Next the expression levels of important enzymes for glucose handling in the livers of ob/ob mice treated with Example 14 was investigated. As seen in FIG. 6B, the mRNA level for GK, the initial step in glycolysis, was increased in treated mice, in line with decreased blood glucose levels. However, in contrast, mRNA for the glycolytic gene LPK was reduced by 30% (FIG. 6B). A similar result for the LPK mRNA was also found in the Slc10a2−/− livers (FIG. 4D). Also, the expression of the gluconeogenic genes G6Pase and PEPCK (FIG. 6B) was analyzed and found that they were both reduced in the inhibitor treated mice, in line with the finding of reduced blood glucose levels in these animals.

Inhibition of Slc10a2 reduces Akt and Mek1/2 activity in ob/ob mouse liver

To further study the molecular mechanisms underlying the observed changes in hepatic gene transcription in the inhibitor treated ob/ob mice, the activity of major kinase signal pathways known to be important in hepatic regulation of both glucose metabolism and lipogenesis was examined. Akt has been demonstrated to be a crucial component in regulating the hepatic response to insulin and other circulating factors with capacity to favour glycolysis and lipogenesis, and inhibit luconeogenesis upon food intake. The induction of SREBP 1c transcription by insulin is dependent on an Akt pathway (27). When Akt actvity was evaluated by phosphospecific antibodies, a reduced serine 473 phosphorylation was noted in liver lysates from inhibitor-treated animals (FIG. 7A). Another kinase pathway activated by the insulin receptor and the FGF receptor 4/beta-Klotho complex, known as the FGF15 receptors, is the Mek1/2—Erk1/2 pathway. Interestingly, it was found that also these important kinases displayed reduced activation in the ob/ob livers upon Slc10a2 inhibitor treatment.

### Conclusions

Interruption of the enterohepatic circulation of bile acids (BA) increases cholesterol metabolism, thereby stimulating hepatic cholesterol synthesis from acetate. The subsequent reduction of the hepatic acetate pool may alter triglyceride (TG) and glucose metabolism. This was explored in mice genetically deficient of the ileal apical sodium BA transporter (Slc10a2) and in ob/ob mice treated with Example 14 an inhibitor of Slc10a2. Plasma TG levels were reduced in Slc10a2 deficient mice, and when challenged with a sucrose-rich diet, they displayed a reduced response in hepatic TG production as observed from the mRNA levels for key enzymes in fatty acid synthesis, ACL, ACC, FAS, SCD1. This effect was paralleled by a diminished induction of mature SREBP1c. Consistently, pharmacologic inhibition of Slc10a2 in diabetic ob/ob mice reduced serum glucose, insulin and TGs, as well as hepatic mRNA levels of SREBP1c and its target genes ACC and FAS. These responses are contrary to those reported following treatment of ob/ob or db/db mice with a BA binding resin. Moreover, when key metabolic signal transduction intermediates in the liver were investigated, it was found that the Mek1/2-Erk1/2 pathway together with Akt were blunted in ob/ob mice after treatment with the Slc10a2 inhibitor. It is concluded that abrogation of Slc10a2 reduces hepatic SREBP1c activity and serum TGs, and in the diabetic ob/ob model also reduces glucose and insulin levels. Hence, targeting Slc10a2 may be a strategy to treat hypertriglyceridemia and diabetes.

### Example 17

In a Phase Ilb, Double-blind, Randomised, Placebo-controlled, Multi-centre, Dose-finding,

Efficacy and Safety of a Range of Doses of the substance according to Example 14 in Patients with Chronic Idiopathic Constipation in addition also have glucose values above Upper limit of normal (ULN), treated for 56 days. The patient was a male or non-pregnant female 20 years of age and 80 years of age with body mass index (BMI) 18.5 but <35.

When baseline value of glucose concentration was compared with end of treatment concentration the patients with higher values of glucose than ULN showed a significant reduction with 36% at the dose 10 mg after 56 days of treatment with Example 14 (Table 2, FIG. 8). Contradictory patients with lower or equal value to ULN did not show any significant change with the substance according to Example 14.

TABLE 2
Glucose concentrations in mg/dL

| | | Placebo | 10 mg |
|---|---|---|---|
| ≦ULN | | | |
| n | | 26 | 25 |
| Baseline | Mean ± SD | 85.8 ± 11.37 | 91.3 ± 7.52 |
| End of Treatment | Mean ± SD | 90.7 ± 16.62 | 90.6 ± 12.96 |
| | % change | +6% | −1% |
| | p-value | | 0.653 |
| >ULN | | | |
| n | | 7 | 4 |
| Baseline | Mean ± SD | 128.1 ± 14.87 | 129.8 ± 7.4 |
| End of Treatment | Mean ± SD | 104.4 ± 14.88 | 82 ± 10.23 |
| | % change | −18.5% | −36.9 |
| | p-value | | 0.013 |

p-values were obtained from an analysis of covariance with treatment group, demographic group and treatment-by-demographic group as fixed factors and baseline value as a covariate in the model. No adjustment for multiple comparisons was made.

Thus, the substance according to Example 14 turned out to be very specifically effective in lowering high glucose values in plasma, whereas normal values were almost not affected.

### Example 18

A formulation for delayed release of the IBAT inhibitor having the following composition can be prepared:

| Substance | amount/capsule (mg) |
|---|---|
| IBAT inhibitor compound Example 14) | 10 |
| Non pareil spheres | 500 |
| Ethyl cellulose | 2 |
| Hydroxypropylmethyl cellulose | 10 |
| Eudragit L100-55 | 25 |
| Triethylcitrate | 2.4 |

The active drug can be dissolved together with ethyl cellulose and hydroxypropyl cellulose in ethanol 99%. The mixture can then be sprayed onto the non-pareil spheres in a fluidized bed apparatus. Thereafter, the pellets can be dried and aerated to remove residual ethanol. The Eudragit L100-55 dispersion with addition of triethyl citrate can then be sprayed onto the drug beads in a fluidized bed apparatus. Subsequently, the coated beads can be filled in hard gelatine capsules after drying and sieving.

### Example 19

A formulation for delayed release of the IBAT inhibitor having the following composition can be prepared:

| Ingredient | amount/tablet (mg) |
|---|---|
| IBAT inhibitor compound Example 14 | 10 |
| Silicon dioxide | 200 |
| Povidone K-25 | 20 |
| Eudragit FS30D | 30 |
| Microcrystalline cellulose | 250 |
| Sodium stearyl fumarate | 5 |

The active drug can be suspended in water and sprayed onto silicon dioxide cores of a predefined size in a fluidized bed apparatus. The drug pellets can be dried in an oven at 400 C. for 24 h. Thereafter, a layer of Povidone K-25 can be applied on the beads from an ethanolic solution in a fluidized bed apparatus. A final coat of Eudragit FS30D dispersion can be applied thereafter in a fluidized bed. The coated beads can be mixed with microcrystalline cellulose and sodium stearyl fumarate in a mixer and subsequently compressed to tablets.

### Example 20

An IBAT inhibitor—colesevelam combination tablet with immediate release of the IBAT inhibitor and colon release of the bile acid binder having the following composition can be prepared:

| | |
|---|---|
| Ingredient | amount/tablet (mg) |

Core

| | |
|---|---|
| Colesevelam hydrochloride | 400 |
| Microcrystalline cellulose | 150 |
| Hydroxypropyl methyl cellulose | 50 |
| Colloidal silicon dioxide | 10 |
| Magnesium stearate | 5 |

Colon release layer

| | |
|---|---|
| Eudragit FS30D | 60 |
| PlasACRYL T20 | 6 |

IBAT inhibitor layer

| | |
|---|---|
| IBAT inhibitor Example 14 | 7 |
| Hydroxypropylmethyl cellulose | 12 |
| Croscarmellose sodium | 6 |

Protective coating

| | |
|---|---|
| Hydroxypropylmethyl cellulose | 12 |
| Polyethylene glycol | 2 |

Colesevelam hydrochloride, microcrystalline cellulose and colloidal silicon dioxide were mixed and granulated with hydroxypropyl methyl cellulose dissolved in water. The granules were dried and mixed with magnesium stearate and compressed to tablets. The EUDRAGIT FS3OD dispersion and water were stirred into the PIasACRYL T20 and sprayed onto the core tablets using a suitable coating machine. The IBAT inhibitor coating suspension was prepared by mixing the IBAT inhibitor, hydroxypropyl methyl cellulose and croscarmellose sodium in water and sprayed onto the tablet cores with the colon release layer using a suitable coating machine. Finally the protective coating solution of hydroxypropylmethyl cellulose and polyethylene glycol was sprayed onto the tablets using a suitable coating machine.

### Example 21

A Colesevelam colon release tablet having the following composition can be prepared:

| | |
|---|---|
| Ingredient | amount/tablet (mg) |

Core

| | |
|---|---|
| Colesevelam hydrochloride | 400 |
| Microcrystalline cellulose | 150 |
| Hydroxypropyl methyl cellulose | 50 |
| Colloidal silicon dioxide | 10 |
| Magnesium stearate | 5 |

Colon release layer

| | |
|---|---|
| Amylose | 30 |
| Eudragit S100 | 60 |
| Triethylcitrate | 6 |
| Glycerolmonostearate | 3 |

Colesevelam hydrochloride, microcrystalline cellulose and colloidal silicon dioxide were mixed and granulated with hydroxypropyl methyl cellulose dissolved in water. The granules were dried and mixed with magnesium stearate and compressed to tablets. Amylose, Eudragit 100, triethylcitrate and glycerolmonosterate were dissolved in suitable solvents and sprayed onto the tablet cores using a suitable coating machine.

1. A compound having IBAT inhibitory effect chosen of formula (I): 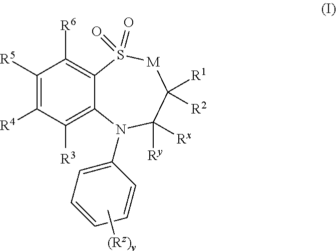
wherein:
M is CH₂, NH
One of le and R² are selected from hydrogen or Cᵢ₋₆alkyl and the other is selected from C₁₋₆alkyl;
R^{x} and R^{y} are independently selected from hydrogen, hydroxy, amino, mercapto, C₁₋₆alkyl, C₁₋₆alkoxy, N-(C₁₋₆alkyl)amino, N,N-(C ₁₋₆alkyl)₂amino, C₁₋₆alkylS(O)ₐ wherein a is 0 to 2
R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkanoyl, C₁₋₆alkanoyloxy, N-(C₁₋₆alkyl)amino, N,N-(C ₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C ₁₋₆alkyl)₂sulphamoyl;
v is 0-5;
one of R⁴ and R⁵ is a group of formula (IA): 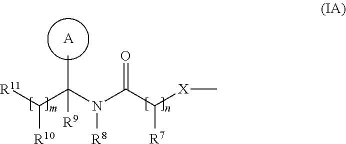
R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;
X is —O—, —N(R^{a})-, -S(O)_{b}- or —CH(Ra)-; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R^{N} is hydrogen, C₁₋₄alkyl, carbocyclyl or heterocyclyl; wherein R¹⁰ is optionally substituted by one or more substituents selected from R¹⁹;
R^{H} is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein Rc and R^{d} are independently selected from C₁₋₆alkyl;
or R¹¹ is a group of formula (IB) or (IC): 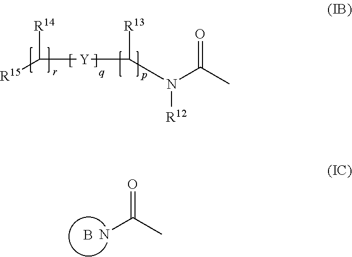
wherein:
Y is —N(R¹¹)-, —N(Rⁿ)C(O)-, —N(Rⁿ)C(O)(CR^{s}OᵥN(Rⁿ)C(O)-, -O-, and -S(O)a-; wherein a is 0-2, v is 1-2, R^{s} and R^{t} are independently selected from hydrogen or C₁₋₄alkyl optionally substituted by R²⁶ and Rⁿ is hydrogen or C₁₋₄alkyl;
R¹² is hydrogen or C₁₋₄alkyl;
R¹³ and R¹⁴ are independently selected from hydrogen, C₁₋₄alkyl, carbocyclyl or heterocyclyl; and when q is 0, R¹⁴ may additionally be selected from hydroxy wherein R¹³ and R¹⁴ may be independently optionally substituted by one or more substituents selected from R²⁰;
R¹⁵ is carboxy, sulpho, sulphino, phosphono, —P(O)(OR^{e})(00, —P(O)(OH)(OR^{e}), —P(O)(OH)(R^{e}) or —P(O)(OR^{e})(R^{f}) wherein R^{e} and R^{f} are independently selected from C₁₋₆alkyl;
p is 1-3; wherein the values of R¹³ may be the same or different;
q is 0-1;
r is 0-3; wherein the values of R¹⁴ may be the same or different;
m is 0-2; wherein the values of R¹⁰ may be the same or different;
n is 1-3; wherein the values of R⁷ may be the same or different;
Ring B is a nitrogen linked heterocyclyl substituted on carbon by one group selected from R²³, and optionally additionally substituted on carbon by one or more R²⁴; and wherein if said nitrogen linked heterocyclyl contains an —NH— moiety, that nitrogen may be optionally substituted by a group selected from R²⁵;
R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;
R¹⁹, R²⁰, R²⁴ and R²⁶ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, benzyloxycarbonylamino, sulpho, sulphino, amidino, phosphono, —P(O)(OR^{a})(00, —P(O)(OH)(OR^{a}), —P(O)(OH)(R^{a}) or —P(O)(ORa)(R^{h}), wherein R^{a} and R^{b} are independently selected from C₁₋₆alkyl; wherein R¹⁹, R20, R²⁴and R²⁶ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
R²³ is carboxy, sulpho, sulphino, phosphono, —P(O)(OR^{g})(OR^{h}), —P(O)(OH)(OR^{g}), —P(O)(OH)(R^{g}) or —P(O)(OR^{g})(R^{h}) wherein Rg and R^{h} are independently selected from C₁₋₆alkyl;
R²⁵ is selected from C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, 1V,N-(C₁₋₆alkyl)carbamoyl, benzyl, benzyloxycarbonyl, benzoyl and phenylsulphonyl;
or a pharmaceutically acceptable salt, solvate, solvate of such a salt or a prodrug thereof for use in prophylaxis and treatment of metabolic syndrome, obesity, disorders of fatty acid metabolism, glucose utilization disorders, disorders in which insulin resistance is involved, diabetes mellitus, type 1 and type 2 diabetes.

2. The compound according to claim 1, chosen from a compound of Formula II: 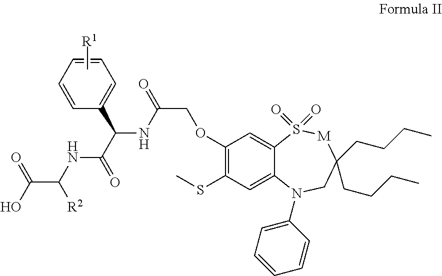
wherein:
M is CH₂ or NH;
R¹ is H or hydroxyl; and
R² is H, CH₃, CH₂CH₃, CH₂CH₂CH₃, CH₂CH₂CH₂CH₃, CH(CH₃)₂, CH₂CH(CH₃)₂, CH(CH₃)CH₂CH₃, CH₂OH, CH₂OCH₃, CH(OH)CH₃, CH₂SCH₃, CH₂CH₂SCH₃, or a pharmaceutically acceptable salt, solvate, solvate of such a salt or a prodrug thereof.

3. The compound according any of claims 1-2, chosen from:
1,1-Dioxo-3,3-dibutyl-5-phenyl-7-methylthio-8-(N-{(R)-α-[N-(carboxymethyl)carbamoyl] benzyl} carbamoylmethoxy)-2,3,4,5-tetrahydro-1,2,5-benzothiadiazepine,
1,1-Dioxo-3, 3-dibutyl-5-phenyl-7-methylthio-8-(N-{(R)-α-[N′-((S)-1-carboxyethyl) carbamoyl] benzyl} carbamoylmethoxy)-2, 3,4, 5-tetrahydro-1, 5-benzothiazepine,
1,1 -Dioxo-3 ,3 -dibutyl-5 -phenyl-7-methylthio-8-(N-{ (R)-α-[N-((S)-1 -carboxypropyl) carbamoyl]benzyl} carbamoylmethoxy)-2,3 ,4,5 -tetrahydro-1,2,5 -benzothiadiazepine,
1,1 -Dioxo-3 ,3 -dibutyl-5 -phenyl-7-methylthio-8-(N-{(R)-α-[N-((R)-1 -carboxy-2-methylthioethyl)carbamoyl]benzyl} carbamoylmethoxy)-2,3 ,4,5 -tetrahydro-1,2,5 -benzothiadiazepine,
1,1 -Dioxo-3 ,3 -dibutyl-5 -phenyl-7-methylthio-8-(N-{ (R)-α-[N-((S)-1 -carboxypropyl) carbamoyl]-4-hydroxybenzyl} carbamoylmethoxy)-2,3 ,4,5 -tetrahydro-1,2,5 -benzothiadiazepine
1,1 -Dioxo-3 ,3-dibutyl-5-phenyl-7-methylthio-8-(N-{(R)-α-[N-((R)-1 -carboxy-2-methylthio-ethyl)carbamoyl]-4-hydroxybenzyl} carbamoylmethoxy)-2,3 ,4,5 -tetrahydro-1,2,5 -benzothiadiazepine,
1,1 -Dioxo-3 ,3 -dibutyl-5 -phenyl-7-methylthio-8-(N-{ (R)-α-[N-((S)-1 -carboxy-2-methylpropyl)carbamoyl]benzyl} carbamoylmethoxy)-2,3 ,4,5 -tetrahydro-1,2,5 -benzothiadiazepine,
1,1 -Dioxo-3 ,3-dibutyl-5-phenyl-7-methylthio-8-(N-{ (R)-α-[N-((S)-1 -carboxy-2-(R)-hydroxypropyl)carbamoyl]-4-hydroxybenzyl} carbamoylmethoxy)-2,3 ,4,5 -tetrahydro-1,2,5 -benzothiadiazepine,
1,1 -Dioxo-3 ,3 -dibutyl-5 -phenyl-7-methylthio-8-(N-{ (R)-α-[N-((S)-1 -carboxybutyl) carbamoyl]-4-hydroxybenzyl} carbamoylmethoxy)-2,3 ,4,5 -tetrahydro-1,2,5 -benzothiadiazepine,
1,1 -Dioxo-3 ,3 -dibutyl-5 -phenyl-7-methylthio-8-(N-{ (R)-α-[N-((S)-1 -carboxyethyl) carbamoyl]benzyl} carbamoylmethoxy)-2,3 ,4,5 -tetrahydro-1,2,5 -benzothiadiazepine,
1,1-dioxo-3, 3-dibutyl-5-phenyl-7-methylthio-8-(N-{(R)-α-[N′-((S)-1-carboxypropyl) carbamoyl]-4-hydroxybenzyl} carbamoylmethoxy) -2, 3,4, 5-tetrahydro-1, 5-benzothiazepine;
1,1-Dioxo-3,3-dibutyl-5-phenyl-7-methylthio-8-(N-{(R)-α-[N-((S)-1-carboxyethyl)carbamoyl]-4-hydroxybenzyl} carbamoylmethoxy)-2,3 ,4,5 -tetrahydro-1,2,5 -benzothiadiazepine,
1,1 -Dioxo-3 ,3 -dibutyl-5 -phenyl-7-methylthio-8-(N-{ (R)-a-[N-((S)-1 -carboxy-2-methylpropyl)carbamoyl]-4-hydroxybenzyl} carbamoylmethoxy)-2,3 ,4,5 -tetrahydro-1,2,5 -benzothiadiazepine, and
1,1-Dioxo-3, 3-dibutyl-5-phenyl-7-methylthio-8-(N-{ (R)-1′-phenyl-1′-[N′-(carboxymethyl) carbamoyl] methyl} carbamoylmethoxy)-2, 3,4, 5-tetrahydro-1, 5-benzothiazepine, or a pharmaceutically acceptable salt, solvate, solvate of such a salt or a prodrug thereof

4. A composition comprising a compound according to claim 1.

5. The composition according to claim 4, further comprising at least one other active substance.

6. The composition according to claim 5 wherein the at least one other active substance is chosen from other IBAT inhibitors, enteroendocrine peptides and enhancers thereof, dipeptidyl peptidase-IV inhibitors, biguanidies; incretin mimetics, thiazolidinones, PPAR agonists, HMG Co-A reductase inhibitors, bile acid binders and TGR5 receptor modulators, or a pharmaceutically acceptable salt, solvate, solvate of such a salt or a prodrug thereof.

7. The composition according claim 6, wherein the bile acid binder is a resin such as cholestyramine, cholestipol and colesevelam.

8. The composition according to claim 6, wherein the HMG Co-A reductase inhibitor, is chosen from statins.

9. The composition according to claim 6, wherein the at least one other active substance is selected from dipeptidyl peptidase-IV-inhibitors, PPARγ agonists, statins and bile acid binders in any combination.

10. The composition according to any of claims 4-5, further comprising a pharmaceutically acceptable diluent or carrier.

11. A method of treatment and/or prophylaxis of metabolic syndrome, obesity, disorders of fatty acid metabolism, glucose utilization disorders, disorders in which insulin resistance is involved, diabetes mellitus, type 1 and type 2 diabetes in a warm-blooded animal, in need of such treatment and/or prophylaxis which comprises administering an effective amount of a compound according to any of claims 1-2 or a composition according to any of claims 4-5 to the warm-blooded animal.

12. A kit comprising compound according to any of claims 1-2 or a composition according to any of claims 4-5 and optionally also an instruction for use.

13. A method for treating obesity or diabetes comprising contacting the distal ileum of an individual in need thereof with an IBAT inhibitor.

14. The method of claim 13, wherein contacting the distal ileum of an individual in need thereof with an IBAT inhibitor results in one or more of the following in any combination: a) reduces food intake of the individual; b) induces satiety in the individual; c) reduces blood and/or plasma glucose levels in the individual; d) treats a metabolic disorder in the individual; e) reduces the weight of the individual; f) stimulates L-cells in the distal gastrointestinal tract of the individual; g) increases the concentration of bile acids and salts thereof in the vicinity of L-cells in the distal gastrointestinal tract of the indivdual; and h) enhances enteroendocrine peptide secretion of the individual.

15. The method of claim 13, wherein the IBAT inhibitor is not systemically absorbed.

16. The method of claim 13, further comprising administration of a second agent selected from a DPP-IV inhibitor, a biguanide, an incretin mimetic, a thiazolidinone, GLP-1 or an analogue thereof, and a TGR5 agonist.

17. The method of claim 13, further comprising administration of a DPP-IV inhibitor.

18. The method of claim 13, wherein the method reduces food intake in an individual in need thereof.

19. The method of claim 13, wherein the method induces satiety in an individual in need thereof.

20. The method of claim 13, wherein the method reduces the weight of an individual in need thereof.

21. The method of claim 13, wherein the method enhances enteroendocrine peptide secretion in an individual in need thereof.

22. The method of claim 21, wherein the enteroendocrine peptide is GLP-1, GLP-2, PYY, oxyntomodulin, or a combination thereof.

23. The method of claim 13, wherein contacting the distal ileum of an individual in need thereof with an IBAT inhibitor increases the level of GLP-1 in the blood and/or plasma of the individual by from about 2 times to about 7 times the level of GLP-1 in the blood and/or plasma of the individual prior to contacting the distal ileum of the individual with the IBAT inhibitor.

24. The method of claim 13, wherein contacting the distal ileum of an individual in need thereof with an IBAT inhibitor reduces the level of glucose in the blood and/or plasma of the individual by at least 30% compared to the level of glucose in the blood and/or plasma of the individual prior to contacting the distal ileum of the individual with the IBAT inhibitor.

25. The method of claim 13, wherein contacting the distal ileum of an individual in need thereof with an IBAT inhibitor maintains reduced blood and/or plasma glucose levels in the individual for at least 24 hours compared to blood and/or plasma glucose levels in the individual prior to contacting the distal ileum of the individual with the IBAT inhibitor.

26. A method for preventing or treating congestive heart failure, ventricular dysfunction, toxic hypervolemia, polycystic ovary syndrome, inflammatory bowel disease, impaired bowel integrity, short bowel syndrome, gastritis, peptic ulcer, or irritable bowel disease comprising contacting the distal ileum of an individual in need thereof with an IBAT inhibitor.

27. A method for preventing or treating radiation enteritis comprising contacting the distal ileum of an individual in need thereof with an IBAT inhibitor.
